(19) United States
(12) Patent Application Publication
(10) Pub. No.: US 2018/0197628 A1
(43) Pub. Date: Jul. 12, 2018

(54) SYSTEMS, DEVICES, AND METHODS FOR EXPERIENTIAL MEDICATION DOSAGE CALCULATIONS

(71) Applicant: ABBOTT DIABETES CARE INC., Alameda, CA (US)

(72) Inventors: Charles Wei, Fremont, CA (US); Nathan Crouther, San Francisco, CA (US); Roy Tsuchida, San Jose, CA (US); Gary A. Hayter, Oakland, CA (US)

(21) Appl. No.: 15/863,279

(22) Filed: Jan. 5, 2018

Related U.S. Application Data
(60) Provisional application No. 62/445,142, filed on Jan. 11, 2017.

(51) Int. Cl. G16H 20/10; A61B 5/00; A61B 5/145; G16H 20/60; A61J 7/04

(52) U.S. Cl. CPC G16H 20/10; A61B 5/4839; A61J 7/0418; G16H 20/60; A61B 5/14546

(57) ABSTRACT

Systems, devices, and methods for detecting, measuring and classifying meals for an individual based on analyte measurements are described. These results and related information can be presented to the individual to show the individual which meals are causing the most severe analyte response. These results can be organized and categorized based on preselected criteria or previous meals and results so as to organize and present the results in a format with reference to glucose as the monitored analyte. Systems, devices, and methods for determining a medication dose based on experiential data and for menu planning are also described.

### CROSS-REFERENCE TO RELATED APPLICATIONS

The present application claims priority to and the benefit of U.S. Provisional Patent Application Ser. No. 62/445,142, filed Jan. 11, 2017, which is incorporated by reference herein in its entirety for all purposes.

### FIELD

The present subject matter broadly relates to systems, devices, and methods for, among others, the collection of information about analyte levels of certain individuals and information about meals that those individuals consume, and providing a medication dose recommendation based on the collected information.

### BACKGROUND

The increased prevalence of Type 2 diabetes and Metabolic Syndrome over the past few decades has been attributed to changing diet and activity levels. For example, consumption of more readily available high glycemic index (GI) foods cause rapid post-prandial increase of blood glucose and insulin levels, which has a positive association with weight gain and obesity. These conditions can be further traced to an increased risk of developing these and other diseases.

Most people generally understand the importance of their diet. However, in practice, many people struggle with translating this general awareness to their specific food choices. These problems exist primarily because people cannot directly see the impact of their choices. This can lead to misconceptions around food portion size, and understanding of which foods are relatively healthy, and the necessary duration and intensity of activity to maintain good health. These misconceptions can be exacerbated by advertisements, habits, peer pressure, food preferences, and recommendations based on generalizations.

High glucose levels are primarily driven by the consumption of food. The level of post-prandial glucose is related to the amount of carbohydrates and other meal components consumed by the individual, as well as to the individual's physiological response to meals. Each individual's glycemic response can now be tracked and better understood by in vivo analyte (e.g., glucose) monitoring devices. However, a challenge for analysis of this influx of data is to represent the data in a meaningful manner that enables efficient action.

Understanding of the data related to meal selection, and subsequent results, needs to be made both on a clinical basis, as well as a personal basis for both the individual, meal administrator, and/or medical professional to understand and moderate glucose excursions, such as episodes of hyperglycemia.

Prior attempts to implement software for tracking the user's meal consumption and correlating that to the user's analyte data suffer from numerous deficiencies. For example, those prior systems that require the performance of what can be inconvenient and uncomfortable discrete blood glucose measurements, for example, by requiring an individual to perform a finger stick to place a blood sample on a test strip, suffer from a lack of a sufficient number of data points to adequately determine a glycemic response to the meal. For example, the individual may perform the discrete blood glucose measurement at a time before or after the time when the user's glycemic response peaks, making it difficult to accurately characterize the glycemic response and to compare meals based on the glycemic response. Also, this deficiency in data points makes it extremely difficult to automatically detect the occurrence of a meal event in the user's analyte data. Thus, such systems place significant reliance upon manual logging of meals by the user.

Prior art systems that seek to detect meal events based simply on the existence of a rise in glucose levels, such as US 2003/0208113, are inadequate because they fail to take into account the user's prior meal history and thus can overestimate the number of meals the user has consumed.

When consuming meals, many diabetics must administer a dose of medication such as insulin to compensate for the anticipated glycemic rise that occurs. This dose is often referred to as a meal bolus because it is a one time injection or infusion for the purpose of compensating for a meal. Determining the amount of insulin to be administered can be complicated and difficult. The determination process typically entails using a prior art bolus calculator that relies on a number of parameters such as the user's insulin sensitivity, the user's insulin on-board, and the amount of carbohydrates in the meal to determine the appropriate dosage. The carbohydrate content for home-cooked meals can be particularly difficult to determine as it is often based on the amount of each individual ingredient in the recipe and may require the user to make estimates based on weight of various portions of the meals. It also requires a carbohydrate determination to be made for each part of the meal. For example, in the case of a dinner including meat, casserole, and a vegetable, carbohydrate content must be determined for each separately and then summed together for entry into the bolus calculator. The time and effort required in making such calculations can be particularly burdensome to diabetics and often result in the diabetic guessing as to the carbohydrate content.

Thus, improved systems, devices, and methods for meal information collection, meal assessment and detection, correlation to analyte levels, and medication dose determination are needed.

### SUMMARY

Provided herein are example embodiments of systems, devices, and methods for detecting, measuring, and classifying meals for a human individual in relation to that individual's analyte measurements. These individuals can be those exhibiting or diagnosed with a diabetic condition, those considered as pre-diabetic, those with metabolic syndrome, and even those without diabetes, pre-diabetic, or metabolic syndrome conditions. These individuals can be any person motivated to improve his or her health by adjustment to his or her diet and/or activity practices. Resulting information can be presented to the individual to show which meals or aspects of the meals are causing the most impact on analyte levels. These results can be organized and categorized based on preselected criteria chosen directly by the individual or based upon consultation between the individual and a medical professional.

In many embodiments, the individual's meal-related analyte responses collected by an analyte monitoring system, such as an in vivo analyte monitoring system, can be compared with or linked to meal information to discover common consistencies (or inconsistencies) along with trends therein based on related historical glucose readings and associated algorithms, variables, weights, comparisons, and trends.

Many embodiments disclosed herein are intended to engage the individual by providing direct and timely feedback regarding the individual's meal-related analyte response. In some embodiments, this analyte response can be provided to the individual to characterize the effects of meal consumption.

The present embodiments can be immediately informative and fun to use by the individual, thereby encouraging the individual's experimentation therewith to better understand how their own diet impacts their body's analyte response. The individual can compare and contrast their current and historical analyte data to see their how their own efforts are related to better diet and meal section and how these choices directly affect their health.

Example embodiments are also provided that enable the collection of analyte data from a local population and linking or association of that data with meal information about meals consumed by the local population. The aggregated information can be processed and presented to a user to identify common trends in analyte levels amongst the local population and to identify whether any correlation exists to the common diet of that population. For example, a person with responsibility for dietary selections can utilize the presented information to determine whether meal contents, types, and/or times of administration can or should be adjusted to alleviate an undesirable trend in the aggregate analyte data, such as a reduction in the occurrence of hypoglycemic or hyperglycemic events. These example embodiments have particular suitability for populations in a common living arrangement, examples of which can include senior care facilities, hospitals, rehabilitation centers, schools, dormitories, military compounds or bases, family residences, prison or penal populations, and any other such environment where a group of individuals share one or more meals on a regular basis.

Also provided herein are systems, devices, and methods that enable a user to determine a compensatory medication dose experientially with a software application, based on the recurring nature of meals and activities. The repeated consumption of meals (or performance of activities) can be logged along with the description of the meal (or activity) and the medication dose previously administered to compensate for that meal (or activity). A database can be compiled that categorizes and indexes the meal (or activity) records and the associated doses. Corresponding analyte (e.g., glucose) data can also be associated with each record from the time period corresponding to the consumption of the meal (or performance of the activity). When the same or similar meals are consumed (or activities performed) in the future, the user can utilize the software application to quickly and reliably determine the appropriate medication dosage. Association of the meal (or activity) with analyte data from prior instances where medication dosages were administered can enable the user or an HCP to readily identify beneficial medication titrations to improve future glycemic responses.

Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be depicted schematically rather than literally or precisely.

FIG. 1 is a high level diagram depicting an example embodiment of an analyte monitoring system for real time analyte (e.g., glucose) measurement, data acquisition and/or processing.

FIG. 2A is a block diagram depicting an example embodiment of a reader device configured as a smartphone.

FIG. 2B is a block diagram depicting an example embodiment of a sensor control device.

FIG. 3A is a flow diagram depicting an example embodiment of a method for meal information gathering and assessment.

FIG. 3B is a flow diagram depicting an example embodiment of a method for performing automatic meal detection sensitivity setting adjustments.

FIG. 3C is an example embodiment of a graphical display of a user's analyte data over time, with a superimposition of previous glycemic responses

FIG. 3D is an example of a graph of analyte data over time displaying an example set of data with four different meal events.

FIGS. 4A-E and 5A-F depict example embodiments of graphical user interface display screens.

FIG. 6A is a block diagram depicting an example embodiment of an analyte monitoring system for use with multiple individuals in a local population.

FIG. 6B is a flow diagram depicting an example embodiment of a method of using an analyte monitoring system with multiple individuals in a local population.

FIG. 7 depicts example aggregated analyte data for one or more (or all) individuals in a local population.

FIG. 8 is a block diagram depicting an example embodiment of an analyte monitoring system with an experiential bolus assistant software module.

FIGS. 9A-B depict example embodiments of graphical user interface display screens for meal and exercise entry, respectively, into an experiential bolus assistant displayed via a web-based server.

FIGS. 10A-16 depict example embodiments of graphical user interface display screens for an experiential bolus assistant displayed from an application resident on a mobile computing device.

FIGS. 17A-C are graphical views depicting example embodiments of glucose response reports.

FIG. 18 is a flow diagram depicting an example embodiment of a method of using an experiential bolus assistant software.

FIG. 19 is a block diagram depicting an example embodiment of a menu planning architecture.

### DETAILED DESCRIPTION

Provided herein are example embodiments of systems, devices, and methods for detecting, measuring, and classifying meals for a human individual in relation to that individual's analyte levels. Based on the analyte data collected, meal-related events and their impact on the individual's analyte levels can be further understood and used to modify future meal selection and dietary habits. Moreover, the administration schedule for insulin or other medication can be adjusted based upon the analyte response to particular meals.

Before describing this subject matter in greater detail, it is worthwhile to describe example embodiments of systems, devices, and methods with which the subject matter can be implemented.

A number of systems have been developed for the automatic monitoring of the analyte(s), like glucose, in bodily fluid such as in the blood stream, in interstitial fluid (“ISF”), dermal fluid of the dermal layer, or in other biological fluid. Some of these systems are configured so that at least a portion of a sensor is positioned below a skin surface of a user, e.g., in a blood vessel or in the subcutaneous tissue of a user, to obtain information about at least one analyte of the body.

As such, these systems can be referred to as “in vivo” monitoring systems. In vivo analyte monitoring systems include “Continuous Analyte Monitoring” systems (or “Continuous Glucose Monitoring” systems) that can transmit data from a sensor control device to a reader device continuously without prompting, e.g., automatically according to a schedule. In vivo analyte monitoring systems also include “Flash Analyte Monitoring” systems (or “Flash Glucose Monitoring” systems or simply “Flash” systems) that can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

The in vivo analyte monitoring systems can be differentiated from “in vitro” systems that contact a biological sample outside of the body (or rather “ex vivo”) and that typically include a meter device that has a port for receiving an analyte test strip carrying bodily fluid of the user, which can be analyzed to determine the user's blood sugar level. While in many of the present embodiments the monitoring is accomplished in vivo, the embodiments disclosed herein can be used with in vivo analyte monitoring systems that incorporate in vitro capability, as well has purely in vitro or ex vivo analyte monitoring systems.

The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “reader device” (or simply a “reader”), “handheld electronics” (or a handheld), a “portable data processing” device or unit, a “data receiver,” a “receiver” device or unit (or simply a receiver), or a “remote” device or unit, to name a few. Other devices such as personal computers have also been utilized with or incorporated into in vivo and in vitro monitoring systems.

### Embodiments of In Vivo Monitoring Systems

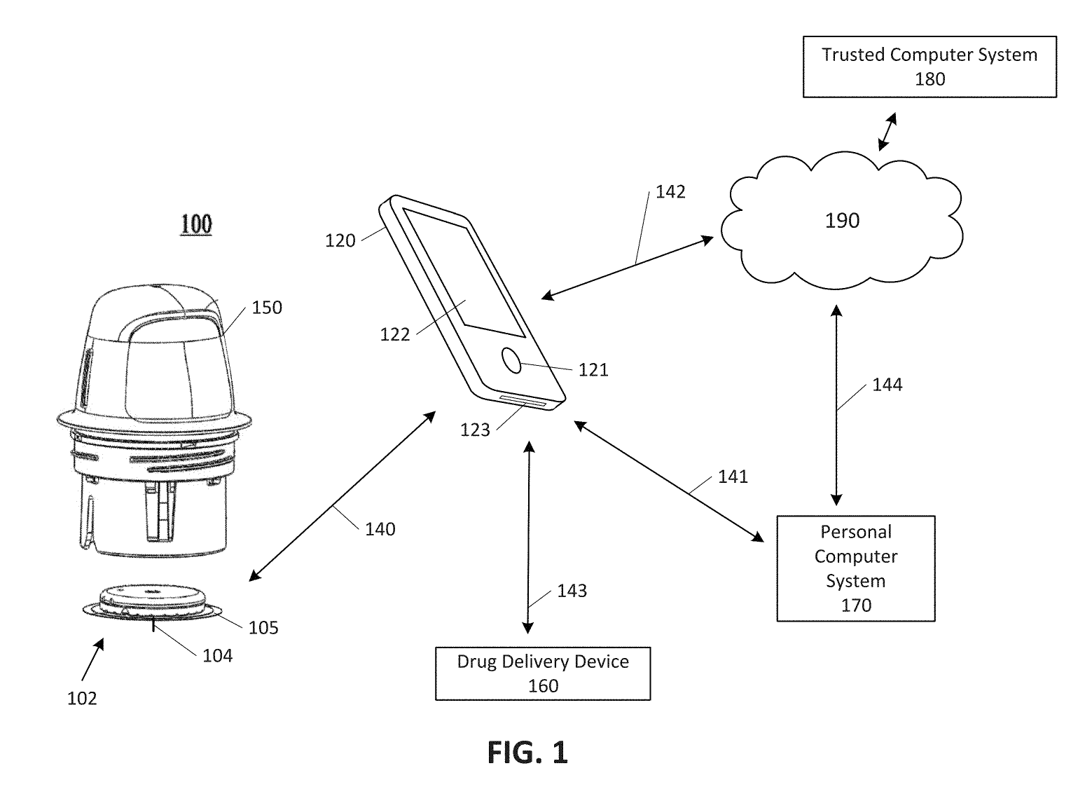

For purpose of illustration, and not limitation, the graphical user interfaces and associated software described herein may be used in connection with an exemplary analyte monitoring system as depicted in FIG. 1. FIG. 1 is an illustrative view depicting an example in vivo analyte monitoring system 100 with which any and/or all of the embodiments described herein can be used. System 100 can have a sensor control device 102 and a reader device 120 that communicate with each other over a local communication path (or link) 140, which can be wired or wireless, and uni-directional or bi-directional. In embodiments where local communication path 140 is wireless, any near field communication (NFC) protocol, RFID protocol, Bluetooth or Bluetooth Low Energy protocol, Wi-Fi protocol, proprietary protocol, or the like can be used, including those communication protocols in existence as of the date of this filing or their later developed variants.

Bluetooth is a well-known standardized short range wireless communication protocol, and Bluetooth Low Energy is a version of the same that requires less power to operate. Bluetooth Low Energy (Bluetooth LE, BTLE, BLE) is also referred to as Bluetooth Smart or Bluetooth Smart Ready. A version of BTLE is described in the Bluetooth Specification, version 4.0, published Jun. 30, 2010, which is explicitly incorporated by reference herein for all purposes. The term “NFC” applies to a number of protocols (or standards) that set forth operating parameters, modulation schemes, coding, transfer speeds, frame format, and command definitions for NFC devices. The following is a non-exhaustive list of examples of these protocols, each of which (along with all of its sub-parts) is incorporated by reference herein in its entirety for all purposes: ECMA-340, ECMA-352, ISO/IEC 14443, ISO/IEC 15693, ISO/IEC 16000-3, ISO/IEC 18092, and ISO/IEC 21481.

Reader device 120 is also capable of wired, wireless, or combined communication, either bidirectional or unidirectional, with either or all of: a drug delivery device 160 over communication path (or link) 143, a personal computer system 170 over communication path (or link) 141, and with a network 190 over communication path (or link) 142. The same wireless protocols described for link 140 can likewise be used for all or part of links 141, 142, and 143.

Reader device 120 can communicate with any number of entities through network 190, which can be part of a telecommunications network, such as a Wi-Fi network, a local area network (LAN), a wide area network (WAN), the internet, or other data network for uni-directional or bi-directional communication. A trusted computer system 180 can be accessed through network 190. In an alternative embodiment, communication paths 141 and 142 can be the same path which can include the network 190 and/or additional networks (e.g., reader 120 communicates with computer 170 through the cloud). All communications over paths 140, 141, 142, and 143 can be encrypted and sensor control device 102, reader device 120, drug delivery device 160, remote computer system 170, and trusted computer system 180 can each be configured to encrypt and decrypt those communications sent and received.

Variants of devices 102 and 120, as well as other components of an in vivo-based analyte monitoring system that are suitable for use with the system, device, and method embodiments set forth herein, are described in U.S. Patent Application Publ. No. 2011/0213225 (the '225 Publication), which is incorporated by reference herein in its entirety for all purposes.

Sensor control device 102 can include a housing 103 containing in vivo analyte monitoring circuitry and a power source (not shown). The in vivo analyte monitoring circuitry can be electrically coupled with an analyte sensor 104 that can extend through an adhesive patch 105 and project away from housing 103. Adhesive patch 105 contains an adhesive layer (not shown) for attachment to a skin surface of the body of the user. Other forms of body attachment to the body may be used, in addition to or instead of adhesive.

Sensor 104 is adapted to be at least partially inserted into the body of the user, where it can make fluid contact with that user's body fluid (e.g., interstitial fluid (ISF), dermal fluid, or blood) and be used, along with the in vivo analyte monitoring circuitry, to measure analyte-related data of the user. Generally, sensor control device 102 and its components can be applied to the body with a mechanical applicator 150 in one or more steps, as described in the incorporated '225 Publication, or in any other desired manner.

After activation, sensor control device 102 can wirelessly communicate the collected analyte data (such as, for example, data corresponding to monitored analyte level and/or monitored temperature data, and/or stored historical analyte related data) to reader device 120 where, in certain embodiments, it can be algorithmically processed into data representative of the analyte level of the user and then displayed to the user and/or otherwise incorporated into a diabetes monitoring regime. In some embodiments, the algorithmic processing of the raw collected measurement data to arrive at data representative of the analyte level of the user and readily displayable (or made displayable) to the user is performed by the processing circuitry of sensor control device 102 prior to transmission to reader device 120.

Various embodiments disclosed herein relate to reader device 120, which can have a user interface including one or more of a display 122, keyboard, optional user interface component 121, and the like. Here, display 122 can output information to the user and/or accept an input from the user (e.g., if configured as a touch screen). Reader device 120 can include one or more optional user interface components 121, such as a button, actuator, touch sensitive switch, capacitive switch, pressure sensitive switch, jog wheel, microphone, or the like. User input can be entered to the user interface in a tactile fashion (e.g., through touching) or a vocal fashion (e.g., into a microphone and processed with speech recognition software). Reader device 120 can also include one or more data communication ports 123 for wired data communication with external devices such as personal computer system 170. Reader device 120 may also include an integrated or attachable in vitro meter, including an in vitro test strip port (not shown) to receive an in vitro analyte test strip for performing in vitro blood analyte measurements.

Drug delivery device 160 is capable of injecting or infusing a drug, such as but not limited to insulin, into the body of the individual wearing sensor control device 102. Like reader device 120, the drug delivery device can include processing circuitry, non-transitory memory containing instructions executable by the processing circuitry, wireless or wired communication circuitry, and a user interface including one or more of a display, touchscreen, keyboard, an input button or instrument, and the like. Drug delivery device 160 can include a drug reservoir, a pump, an infusion tube, and an infusion cannula configured for at least partial implantation into the user's body. The pump can deliver insulin from the reservoir, through the tube, and then through the cannula into the user's body. Drug delivery device 160 can include instructions, executable by the processor, to control the pump and the amount of insulin delivered. These instructions can also cause calculation of insulin delivery amounts and durations (e.g., a bolus infusion and/or a basal infusion profile) based on analyte level measurements obtained directly or indirectly from sensor control device 102. Alternatively, calculations of insulin delivery amounts and durations, and the control of the pump, can be performed by reader device 120 directly. The drug delivery device can be configured to communicate directly with reader device 120 in the form of a closed loop or semi-closed loop system. Alternatively, the drug delivery device can include the functionality of reader device 120 described herein, or vice versa, to arrive at one integrated reader and drug delivery device.

Computer system 170 may be a personal or laptop computer, a tablet, or other suitable data processing device. Computer 170 can be either local (e.g., accessible via a direct wired connection such as USB) or remote to reader device 120 and can be (or include) software for data management and analysis and communication with the components in analyte monitoring system 100. Computer 170 can communicate with network 190 through a bi-directional wired and/or wireless link 144 (e.g., the internet). Operation and use of computer 170 is further described in the '225 Publication incorporated herein by reference. Analyte monitoring system 100 can also be configured to operate with a data processing module (not shown), also as described in the incorporated '225 Publication.

Trusted computer system 180 can be used to perform authentication of sensor control device 102 and/or reader device 120, used to store confidential data received from devices 102 and/or 120, used to output confidential data to devices 102 and/or 120, or otherwise configured. Trusted computer system 180 can include one or more computers, servers, networks, databases, and the like. Trusted computer system 180 can be within the possession of the manufacturer or distributor of sensor control device 102, either physically or virtually through a secured connection, or can be maintained and operated by a different party (e.g., a third party).

Trusted computer system 180 can be trusted in the sense that system 100 can assume that computer system 180 provides authentic data or information. Trusted computer system 180 can be trusted simply by virtue of it being within the possession or control of the manufacturer, e.g., like a typical web server. Alternatively, trusted computer system 180 can be implemented in a more secure fashion such as by requiring additional password, encryption, firewall, or other internet access security enhancements that further guard against counterfeiter attacks or attacks by computer hackers. Trusted computer system 180 can be a secure cloud-based server, for example, that reader device 120, drug-delivery device 160, and/or computer system 170 can remotely upload and/or download data to through network 190 (e.g., the cloud).

The processing of data and the execution of software within system 100 can be performed by one or more processors of reader device 120, computer system 170, and/or sensor control device 102. For example, raw data measured by sensor 104 can be algorithmically processed into a value that represents the analyte level and that is readily suitable for display to the user, and this can occur in sensor control device 102, reader device 120, or computer system 170. This and any other information derived from the raw data can be displayed in any of the manners described above (with respect to display 122) on any display residing on any of sensor control device 102, reader device 120, or computer system 170. The information may be utilized by the user to determine any necessary corrective actions to ensure the analyte level remains within an acceptable and/or clinically safe range.

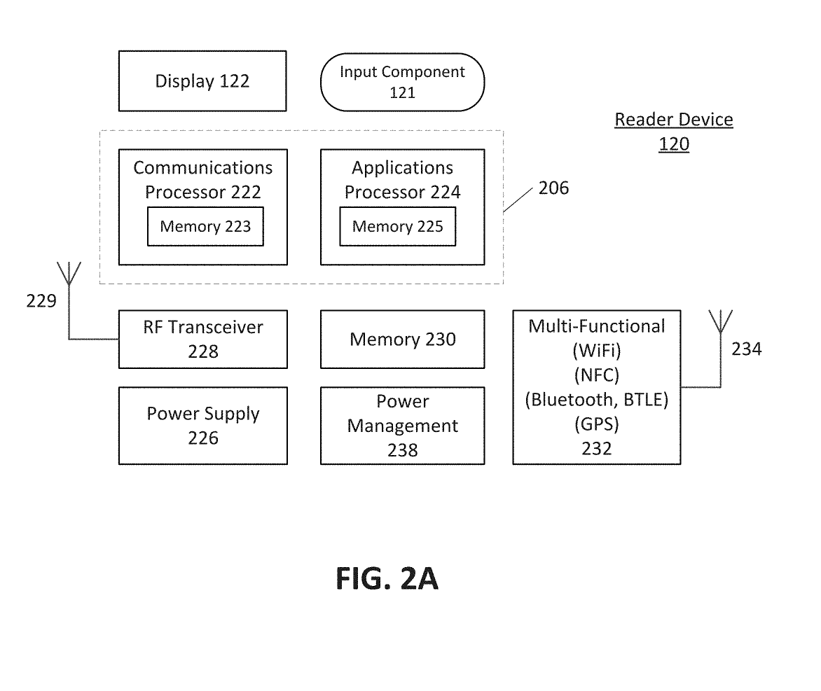

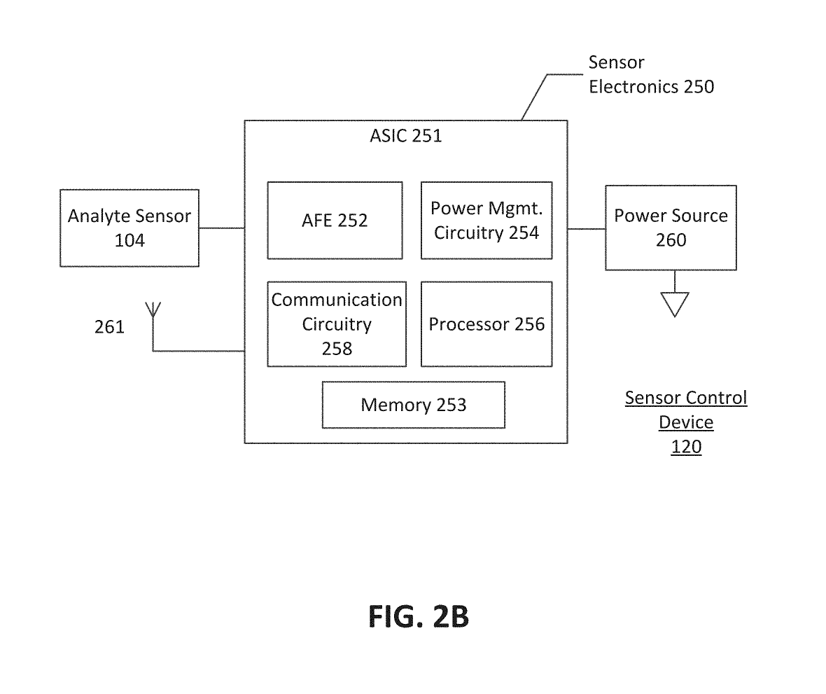

FIGS. 2A-2B depict example embodiments of reader device 120 and sensor control device 102, respectively. As discussed above, reader device 120 can be a mobile communication device such as, for example, a Wi-Fi or internet enabled smartphone, tablet, or personal digital assistant (PDA). Examples of smartphones can include, but are not limited to, those phones based on a WINDOWS operating system, ANDROID operating system, IPHONE operating system, PALM WEBOS, BLACKBERRY operating system, or SYMBIAN operating system, with network connectivity for data communication over the internet or a local area network (LAN).

Reader device 120 can also be configured as a mobile smart wearable electronics assembly, such as an optical assembly that is worn over or adjacent to the user's eye (e.g., a smart glass or smart glasses, such as GOOGLE GLASSES). This optical assembly can have a transparent display that displays information about the user's analyte level (as described herein) to the user while at the same time allowing the user to see through the display such that the user's overall vision is minimally obstructed. The optical assembly may be capable of wireless communications similar to a smartphone. Other examples of wearable electronics include devices that are worn around or in the proximity of the user's wrist (e.g., a watch, etc.), neck (e.g., a necklace, etc.), head (e.g., a headband, hat, etc.), chest, or the like.

FIG. 2A is a block diagram of an example embodiment of a reader device 120 according to various embodiments disclosed herein. In this example, the reader device 120 is in the form of a smartphone, upon which the various software, applications, and graphical user interfaces disclosed herein can reside. Here, reader device 120 includes an input component 121, display 122, and processing hardware 206, which can include one or more processors, microprocessors, controllers, and/or microcontrollers, each of which can be a discrete chip or distributed amongst (and a portion of) a number of different chips. Here, processing hardware 206 includes a communications processor 222 having on-board non-transitory memory 223 and an applications processor 224 having on-board non-transitory memory 225. Reader device 120 further includes an RF transceiver 228 coupled with an RF antenna 229, a memory 230, multi-functional circuitry 232 with one or more associated antennas 234, a power supply 226, and power management circuitry 238. FIG. 2A is an abbreviated representation of the internal components of a smartphone, and other hardware and functionality (e.g., codecs, drivers, glue logic, etc.) can of course be included.

Communications processor 222 can interface with RF transceiver 228 and perform analog-to-digital conversions, encoding and decoding, digital signal processing and other functions that facilitate the conversion of voice, video, and data signals into a format (e.g., in-phase and quadrature) suitable for provision to RF transceiver 228, which can then transmit the signals wirelessly. Communications processor 222 can also interface with RF transceiver 228 to perform the reverse functions necessary to receive a wireless transmission and convert it into digital data, voice, and video.

Applications processor 224 can be adapted to execute the operating system and any software applications that reside on reader device 120 (such as any sensor interface application or analyte monitoring application that includes, e.g., SLL 304), process video and graphics, and perform those other functions not related to the processing of communications transmitted and received over RF antenna 229. Any number of applications can be running on reader device 120 at any one time, and will typically include one or more applications that are related to a diabetes monitoring regime, in addition to the other commonly used applications that are unrelated to such a regime, e.g., email, calendar, weather, etc.

Memory 230 can be shared by one or more of the various functional units present within reader device 120, or can be distributed amongst two or more of them (e.g., as separate memories present within different chips). Memory 230 can also be a separate chip of its own. Memory 230 is non-transitory, and can be volatile (e.g., RAM, etc.) and/or non-volatile memory (e.g., ROM, flash memory, F-RAM, etc.).

Multi-functional circuitry 232 can be implemented as one or more chips and/or components, including communication circuitry, that perform other functions such as local wireless communications (e.g., Wi-Fi, Bluetooth, Bluetooth Low Energy) and determining the geographic position of reader device 120 (e.g., global positioning system (GPS) hardware). One or more other antennas 234 are associated with both the functional circuitry 232 as needed.

Power supply 226 can include one or more batteries, which can be rechargeable or single-use disposable batteries. Power management circuitry 238 can regulate battery charging and power supply monitoring, boost power, perform DC conversions, and the like. As mentioned, reader device 120 may also include one or more data communication ports such as USB port (or connector) or RS-232 port (or any other wired communication ports) for data communication with a remote computer system 170 (see FIG. 1), or sensor control device 102, to name a few.

FIG. 2B is a block schematic diagram depicting an example embodiment of sensor control device 102 having analyte sensor 104 and sensor electronics 250 (including analyte monitoring circuitry). Although any number of chips can be used, here the majority of the sensor electronics 250 are incorporated on a single semiconductor chip 251 that can be, e.g., a custom application specific integrated circuit (ASIC). Shown within ASIC 251 are several high-level functional units, including an analog front end (AFE) 252, power management circuitry 254, processor 256, and communication circuitry 258 (which can be implemented as a transmitter, receiver, transceiver, passive circuit, or otherwise according to the communication protocol). In this embodiment shown in FIG. 2B, both AFE 252 and processor 256 are used as analyte monitoring circuitry, but in other embodiments either circuit can perform the analyte monitoring function. Processor 256 can include one or more processors, microprocessors, controllers, and/or microcontrollers.

A non-transitory memory 253 is also included within ASIC 251 and can be shared by the various functional units present within ASIC 251, or can be distributed amongst two or more of them. Memory 253 can be volatile and/or non-volatile memory. In this embodiment, ASIC 251 is coupled with power source 260, which can be a coin cell battery, or the like. AFE 252 interfaces with in vivo analyte sensor 104 and receives measurement data therefrom and outputs the data to processor 256 in digital form, which in turn processes the data to arrive at the end-result analyte discrete and trend values, etc. This data can then be provided to communication circuitry 258 for sending, by way of antenna 261, to reader device 120 (not shown) where further processing can be performed by, e.g., the sensor interface application. It should be noted that the functional components of ASIC 251 can also be distributed amongst two or more discrete semiconductor chips.

Performance of the data processing functions within the electronics of the sensor control device 102 provides the flexibility for system 100 to schedule communication from sensor control device 102 to reader device 120, which in turn limits the number of unnecessary communications and can provide further power savings at sensor control device 102.

Information may be communicated from sensor control device 102 to reader device 120 automatically and/or continuously when the analyte information is available, or may not be communicated automatically and/or continuously, but rather stored or logged in a memory of sensor control device 102, e.g., for later output.

Data can be sent from sensor control device 102 to reader device 120 at the initiative of either sensor control device 102 or reader device 120. For example, in many example embodiments sensor control device 102 can communicate data periodically in a prompted, unprompted or broadcast-type fashion, such that an eligible reader device 120, if in range and in a listening state, can receive the communicated data (e.g., sensed analyte data). This can be at the initiative of sensor control device 102 because reader device 120 does not have to send a request or other transmission that first prompts sensor control device 102 to communicate. Transmissions can be performed, for example, using an active Wi-Fi, Bluetooth, or BTLE connection. The transmissions can occur according to a schedule that is programmed within device 102 (e.g., about every 1 minute, about every 5 minutes, about every 10 minutes, or the like). Transmissions can also occur in a random or pseudorandom fashion, such as whenever sensor control device 102 detects a change in the sensed analyte data. Further, transmissions can occur in a repeated fashion regardless of whether each transmission is actually received by a reader device 120.

System 100 can also be configured such that reader device 120 sends a transmission that prompts sensor control device 102 to communicate its data to reader device 120. When the prompt is generated manually by the user, this transmission can be referred to as “on-demand” data transfer. An on-demand data transfer can be initiated at the behest of the user via a user interface of reader device 120. For example, if the user wants to check his or her analyte level, the user could perform a scan of sensor control device 102 using an NFC, Bluetooth, BTLE, or Wi-Fi connection. Data exchange can be accomplished using broadcasts only, on-demand transfers only, other manners of transmission, or any combination thereof.

Accordingly, once a sensor control device 102 is placed on the body so that at least a portion of sensor 104 is in contact with the bodily fluid and electrically coupled to the electronics within device 102, sensor derived analyte information may be communicated in on-demand or unprompted fashion from the sensor control device 102 to a reader device 120. On-demand transfer can occur by first powering on reader device 120 (or it may be continually powered) and executing a software algorithm stored in and accessed from a memory of reader device 120 to generate one or more requests, commands, control signals, or data packets to send to sensor control device 102. The software algorithm executed under, for example, the control of processing hardware 206 of reader device 120 may include routines to detect the position of the sensor control device 102 relative to reader device 120 to initiate the transmission of the generated request command, control signal and/or data packet.

Embodiments Associating Analyte Levels with Meal Information

In many embodiments, the subject matter described herein is implemented by a software application program that is stored on and executed by a processor-based device, such as any one of the reader devices, drug delivery devices, or other computing devices described herein. In certain embodiments, the software is implemented as one or more downloadable software applications (“an App”) on a reader device such as a mobile communication device or smartphone.

The software can provide a mechanism for the user to define consumables (e.g., a type of food, type of drink, or portion thereof), in any fashion that is convenient to the user. These consumables will be referred to generally herein as a meal or meals, and these terms are used broadly to denote all types of food and drink.

This software can perform a number of functions related to the collection of meal information and association of that meal information with analyte information collected by in vivo analyte sensor 104 or by in vitro test strip and meter. The software will be generally referred to herein as the “meal monitor application.”

The meal monitor application can allow an individual to log information about each meal that the individual consumes (i.e., each “meal event”), including a photo of the meal. The meal monitor application can associate analyte data from the same general time period where the user's log entry indicated that a meal was consumed.

The meal monitor application can also monitor the user's analyte data and identify when the analyte data changes in a manner indicating or suggesting the occurrence of a potential meal event and seek to associate meal information from the same general time period with that potential meal event. The meal monitor application can prompt the individual for confirmation that the potential meal event occurred and for information describing the meal event. If the individual has already entered meal information, then the system can prompt for additional information about the meal event that the user has not already entered. In some embodiments, if a meal has been detected, and the meal monitor application determines that meal information has already been entered, then the user may not be prompted.

The meal monitor application can also associate the measured analyte response with each meal event and store the result in a non-transitory memory or a database, regardless of whether the analyte response is also classified as or includes an analyte excursion. The meal monitor application can display each meal with its associated analyte (e.g., glucose or other analyte) response to the user, for example, as a list where each meal is sorted by descending degree of, using glucose as an example, glycemic response magnitude. The meal monitor application can display other lists, such as a list of “good” meals made up of meals where the glycemic response magnitude was below a predefined magnitude and/or made up of a number of meals that had the lowest magnitudes of all the meals recorded. Another example is a list of “bad” meals made up of meals where the glycemic response magnitude was above a predefined magnitude and/or made up of a number of meals that had the highest magnitudes of all the meals recorded.

One example embodiment of the glucose response magnitude is the peak glucose value detected from the glucose response after a meal. Methods for determining this peak glucose metric are described further herein. Another example embodiment of the glucose response magnitude is the difference from the peak glucose value after a meal and the glucose value at the start of the detected meal.

In yet another example embodiment, the glucose response magnitude may be determined from a form of “area under the curve.” The system can determine the value of the area with an upper bound set by a trace or curve along (or approximating) the values of the collected glucose data from the start of the detected meal and ending upon or after a) a fixed time such as 6 hours, b) the start of the next detected meal, c) an occurrence when the glucose trace falls below the value of the start of the meal. In one example embodiment, the end point is whichever of the preceding events (a-c) occurs first. The lower bound for the area determination can be the glucose value at the start of the detected meal. The area can be calculated by summing the glucose values between the start and the end (inclusive or exclusive) and multiplying this sum by the total time from start to end, then subtracting the glucose value at the start of the detected meal multiplied by the total time from start to end. Other similar magnitude metrics along these lines can be implemented.

If the user consumes the same or a similar meal on repeated occasions, then a glycemic central tendency (e.g., an average or median glycemic response) can be determined for that meal and that central tendency can be displayed. For example, if the peak of the glycemic response is the metric of choice, then when multiple identical meals have been recorded by the system, the median of the peak values can represent the glycemic response metric for this meal. Other forms of glucose response metrics such as a glucose trace or a parametric fit to the glucose trace, may be used. Alternatively, the glycemic response of every meal can be displayed regardless of whether each meal is the same or similar to another meal on the list or in the database. In yet another embodiment, the meal monitor application may generate a glycemic response as representative of all similar meals; for instance, if the glycemic response is displayed as a trace, the trace representative of all the similar meals may be made up of hourly medians of all of the individual glycemic responses, where time is relative to the start of the meal.

Example embodiments of the meal monitor application can utilize analyte data analysis software or software implementable processes, for example, as disclosed in any of U.S. Patent Publication Nos. 2013/0085358, 2014/0350369 or 2014/0088393, or in Int'l Publ. No. WO 2015/153482, all of which are incorporated herein in their entirety and for all purposes. Example embodiments of this software are collectively referred to herein as the “meal event detector.” The meal event detector can be an algorithm, routine, or other set of instructions (part of or separate from the meal monitor application) that can detect and/or quantify the occurrence of an actual or potential meal event in the individual's monitored analyte data.

Detection of a meal event can include detection of analyte episodes or excursions outside a desired acceptable (e.g., medically recommended) target range in the user, who can be informed by the software that one or both has been detected. Examples of analyte excursions include violation of a low glucose threshold, violation of a high glucose threshold, violation of a rate of change (e.g., increase or decrease) threshold, violation of a glucose median threshold, violation of a glucose variability threshold, and the like.

Some of the meal event detectors described in these incorporated references are described only in terms of identifying analyte excursions outside a desired target range. These embodiments can be extended to the detection of meal events based on the teachings contained within other ones of these incorporated references (e.g., Int'l Publ. No. WO 2015/153482). These embodiments can also be extended to the detection of meal events by specification of a within-target episode, where glucose values are maintained between an upper and lower bound for a period of time. Detection of these episodes can be done by extension of threshold-based episode detection algorithms.

A relatively straightforward example of threshold-based logic includes grouping all consecutive points above or below a threshold into a grouping to be associated with the meal event, which can, in some instances, also be or include an analyte excursion outside of the user's targeted acceptable range.

Very brief episodes (e.g., trends or groupings in the analyte data or outlier values) may not be clinically relevant. Embodiments described herein can manage this challenge by requiring a minimum number of readings and/or a minimum duration and/or a minimum area (e.g., an integration) outside the threshold to consider the episode for analysis as either a meal event or an analyte excursion. An episode failing any of the requirements may be ignored. Software displays and applications for providing upload prompts to an individual are disclosed in the incorporated reference U.S. Patent Publ. No. 2014/088393, which also discloses software applications for providing heuristic meal announcements that can be used herein for analysis and display of data as well as for meal selection.

A virtually limitless catalog of analyte episode types exist (e.g., analyte data occurrences having different characteristics), each of which, if independently clinically relevant, can be used to form the basis for meal reports, meal effects, meal analysis, meal-based treatment, medication administration, and meal selection. Glycemic response and episode characteristics can also be used to classify meals and certify meal-related collected data. Such analysis and results can be presented to the individual, clinical meal supervisor, or physician to make future meal decisions.

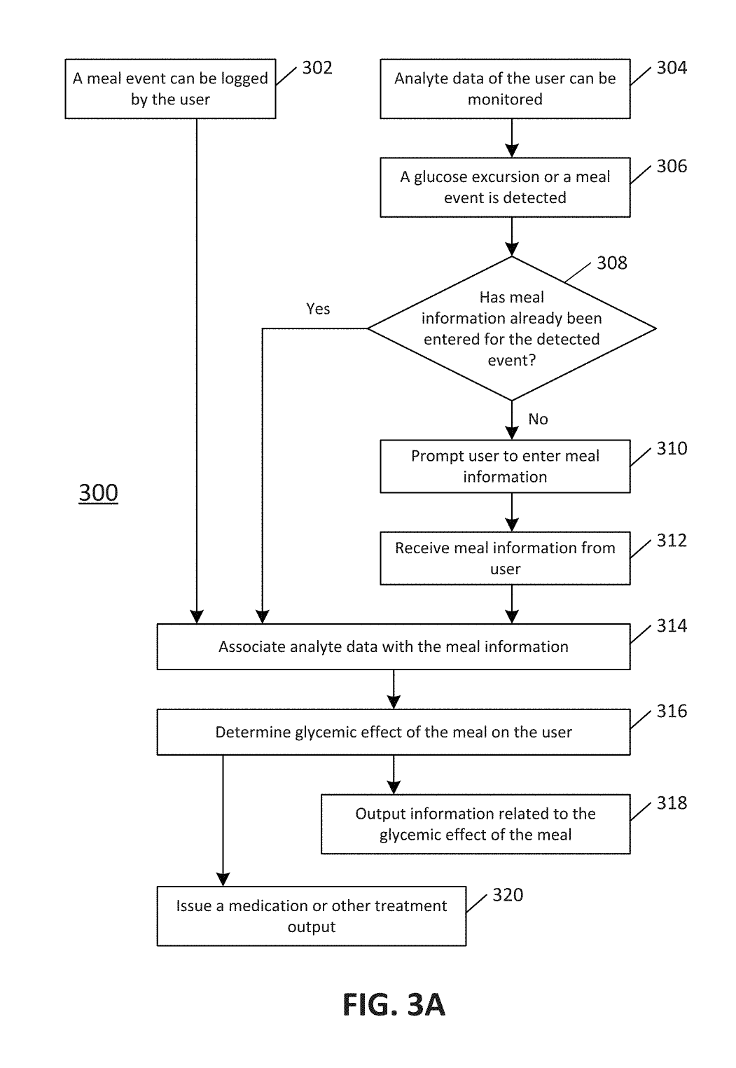

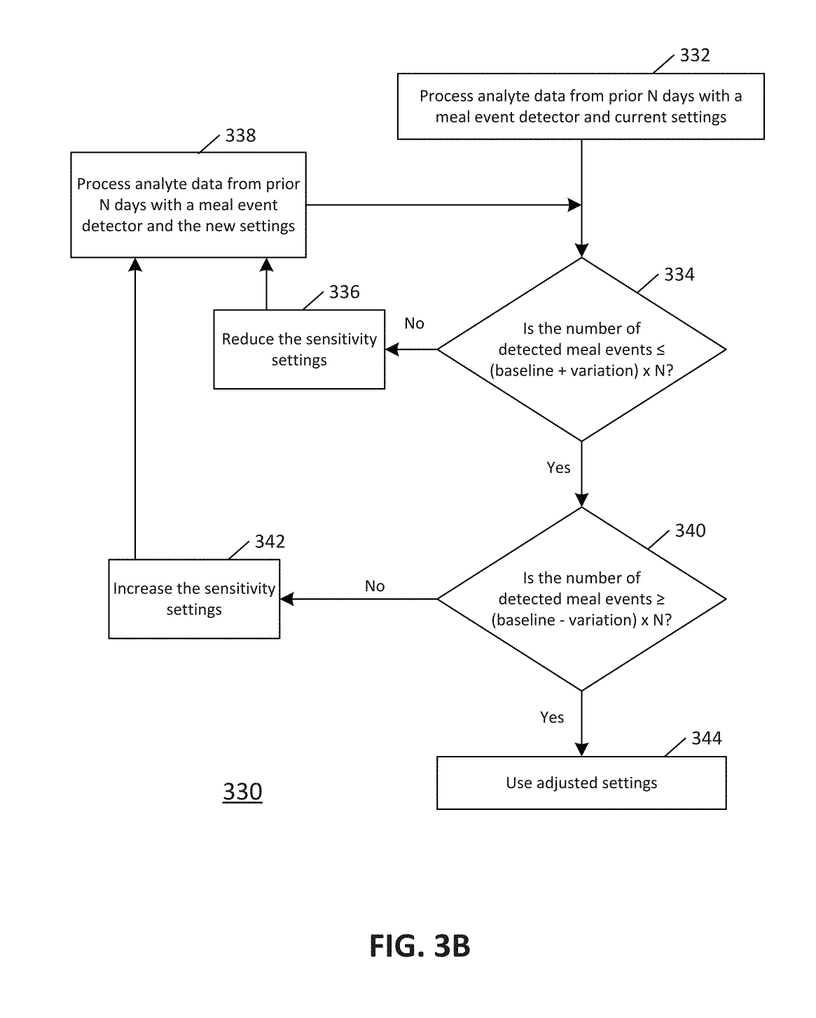

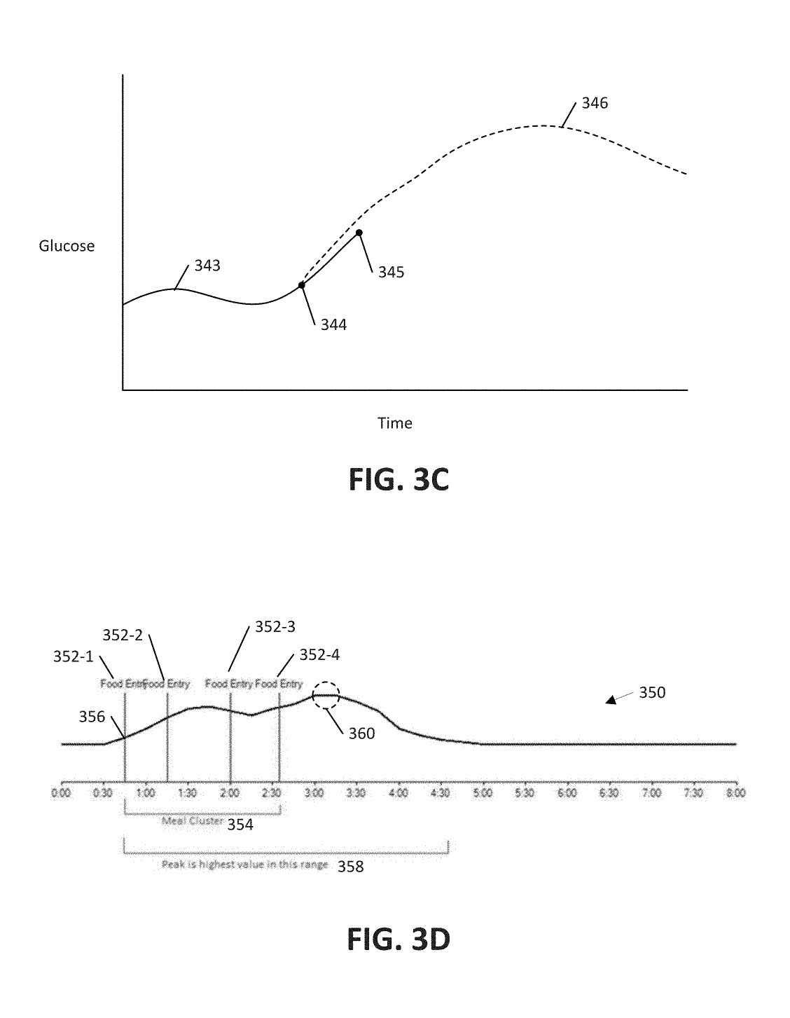

Reference is now made to FIG. 3A, which is a flow diagram depicting an example embodiment of a method 300 for meal information collection, association with analyte data, and determination of glycemic impact of that meal. Method 300 includes acts that may be described as performed by an electronic device, such as reader device 120, drug delivery device 160, or computer system 170 or 180, or processors thereof. The user may be an individual or diabetic sufferer, clinical administrator, medical professional, dietary profession, or other person. By way of example only, method 300 will be described by reference to a diabetic using the meal monitor software as a downloaded app on a reader device 120 configured as a smart phone. For ease of illustration, the monitored analyte in this and other embodiments described below will be glucose, although other analytes can be monitored as well as is noted herein.

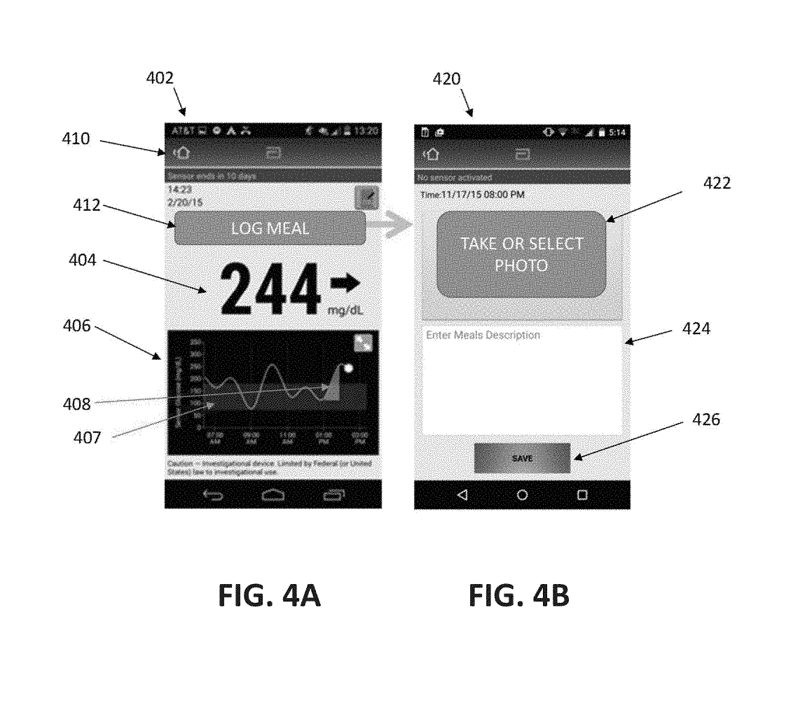

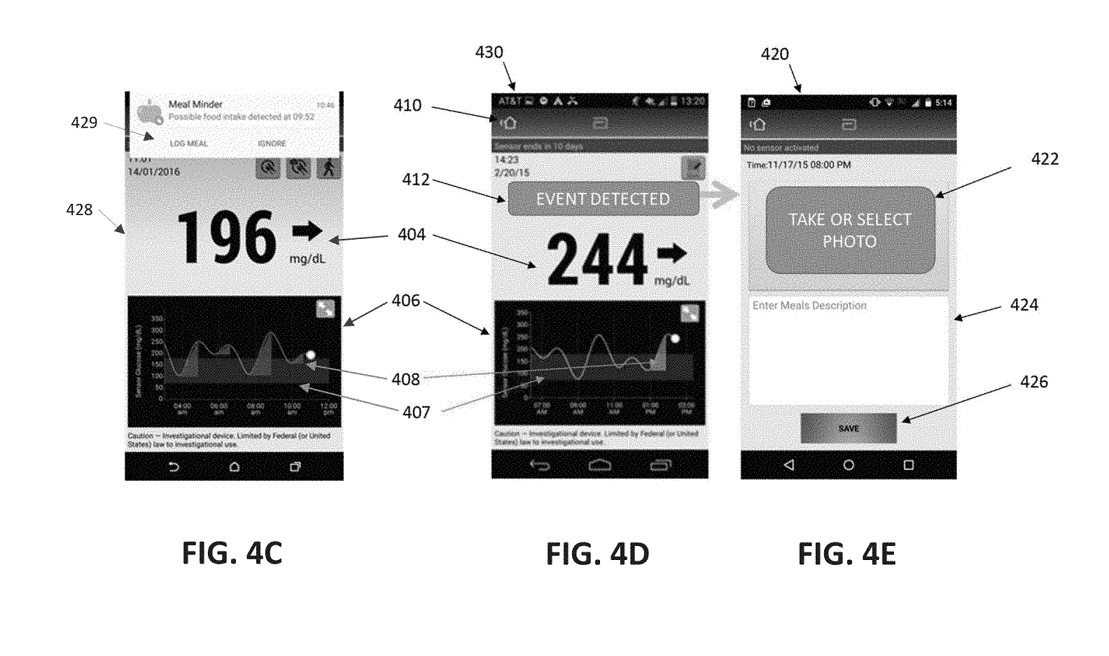

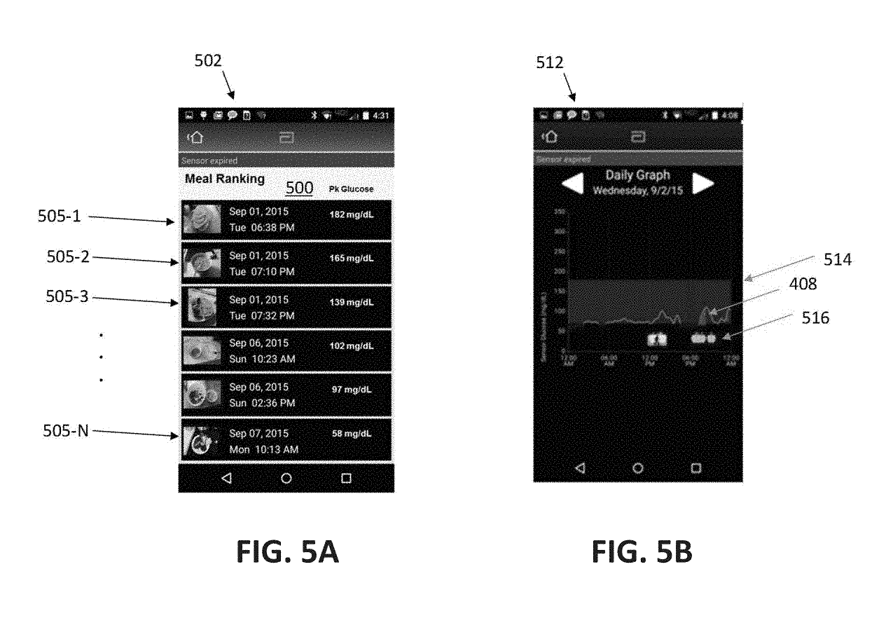

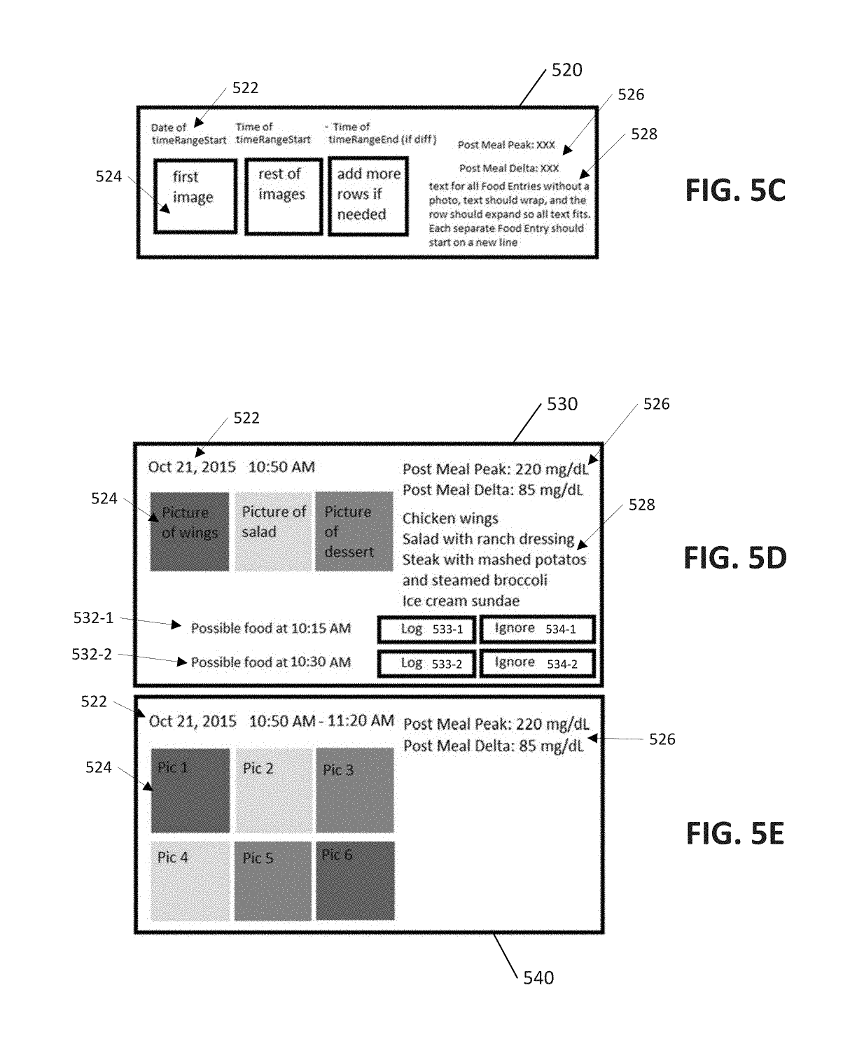

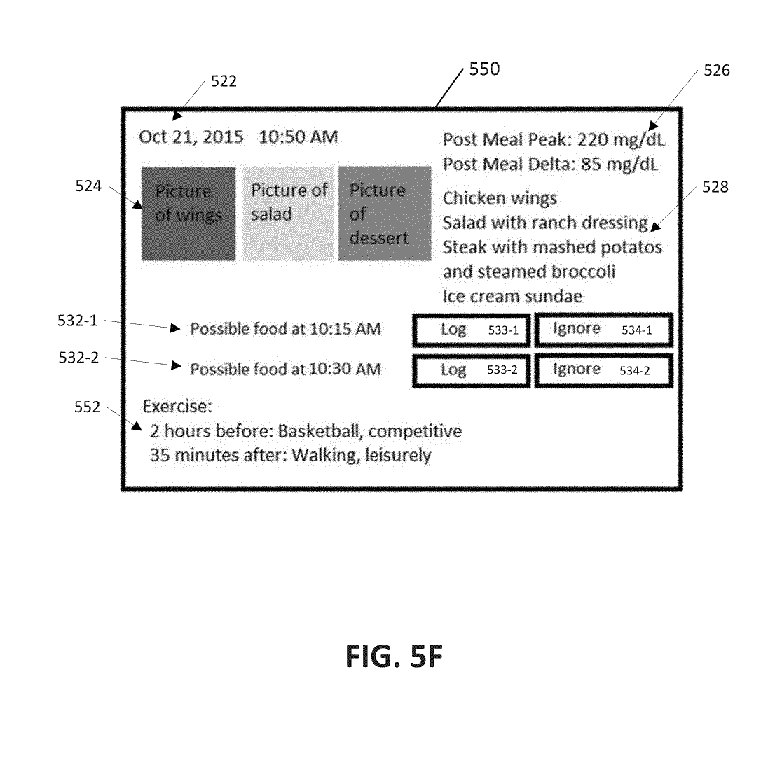

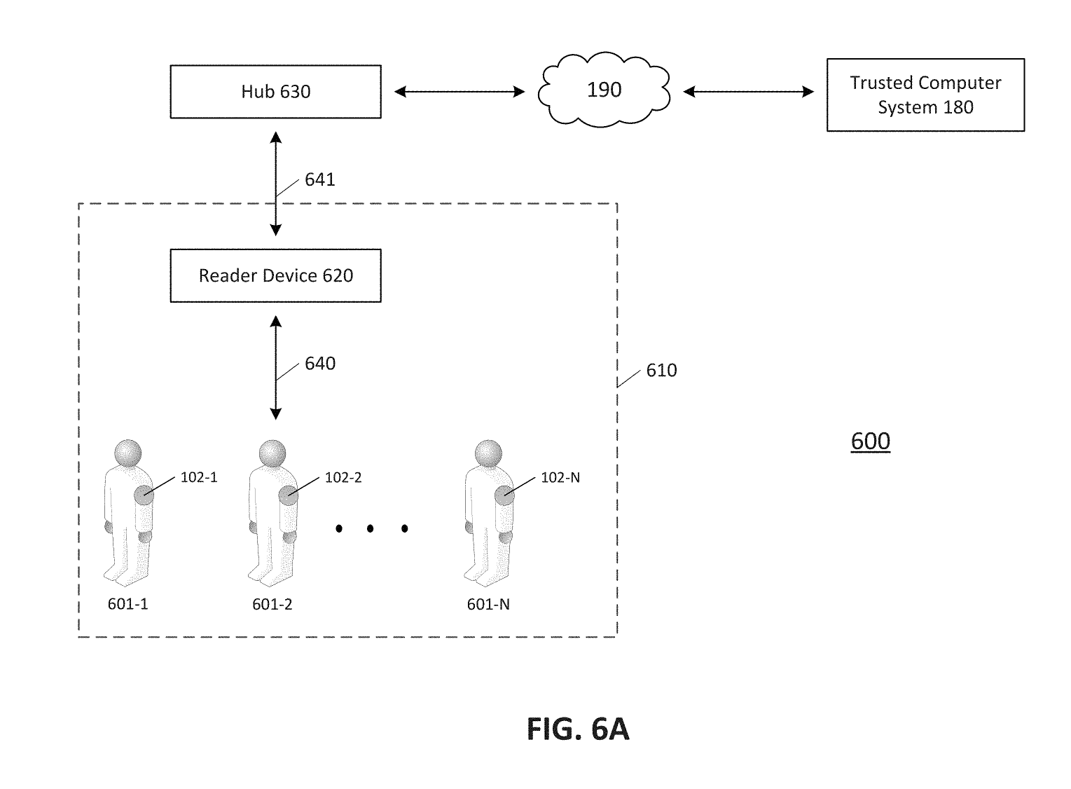

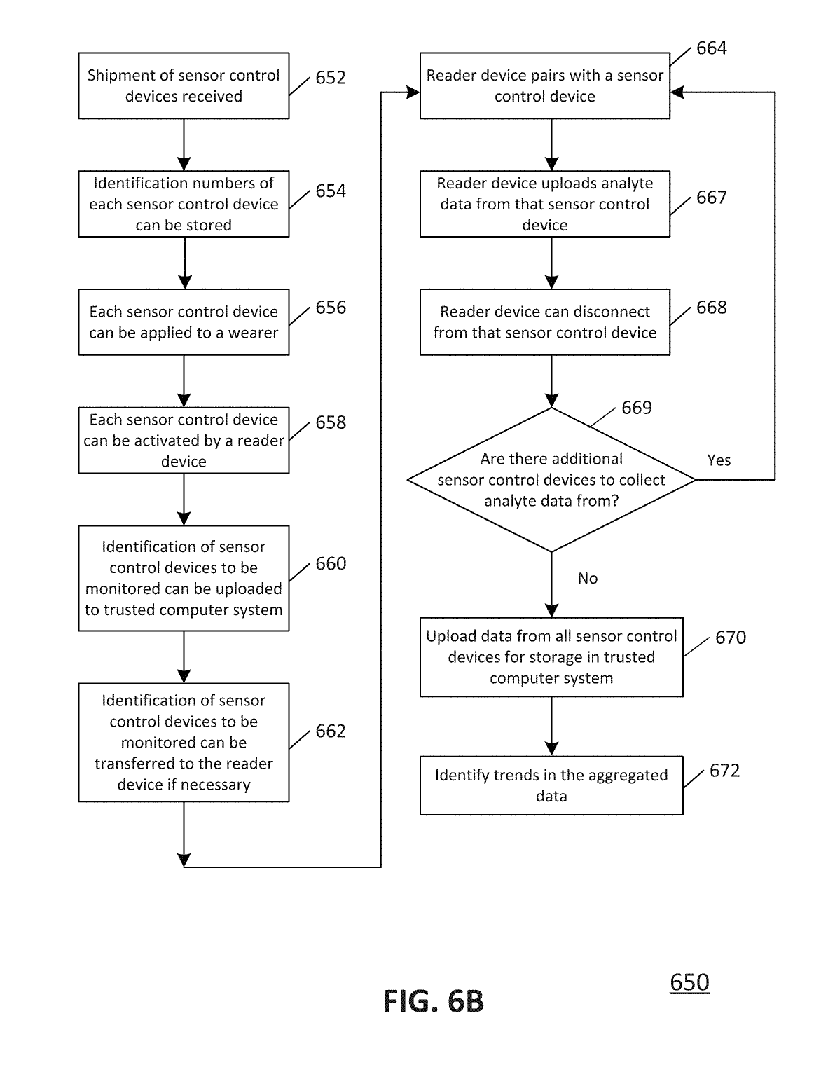

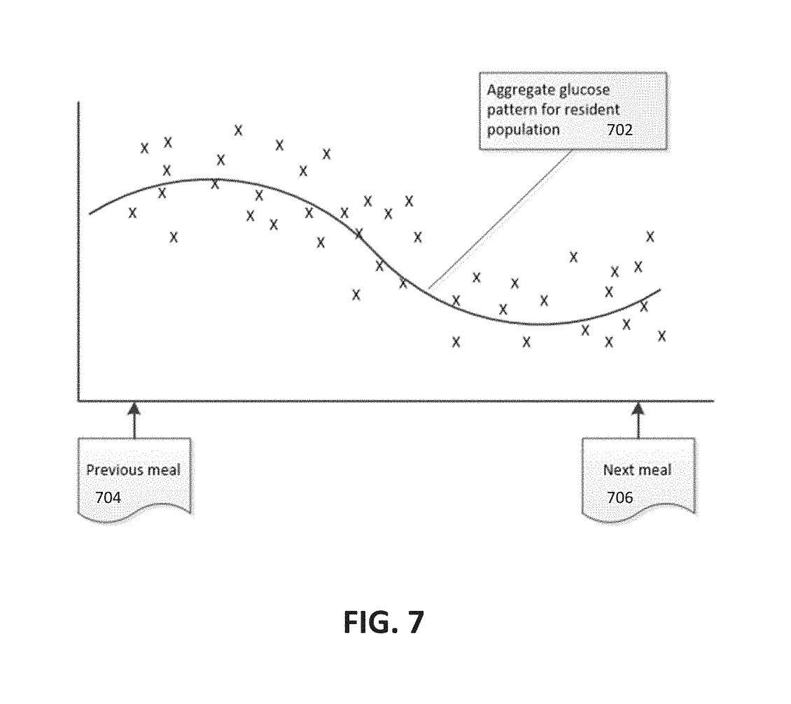

Referring to FIG. 3A, a meal event can be logged by the user at 302. The user can input meal information directly into reader device 120 (via a user interface) at his or her own discretion, before, during, or after consumption of the meal. In some embodiments, the user inputs meal information in response to a reminder generated by the meal monitor application according to a predetermined schedule, which can be set and/or modified by the user. Example embodiments of user interfaces for accepting a manual log by the user are described with respect to FIGS. 4A and 4B.

Analyte data of the user is monitored at 304. This monitoring, in most embodiments, is a continuous process (e.g., a frequently repeated process, automatically or at the discretion of the user) that is ongoing so long as the user is wearing sensor control device 102. Data collected by sensor control device 102 can be transferred to reader device 120 such that the meal monitor application has access to the data. Information indicative of the time at which each analyte data measurement is collected (e.g., a timestamp) can also be transferred to reader device 120. As already described herein, this data transfer can occur in an on-demand fashion (e.g., the performance of a scan by the user), in an automatic prompted or unprompted fashion, or other regularly occurring fashion. Analyte data collected by a discrete blood glucose measurement (e.g., such as the reading of a test strip with a meter) can also be entered into reader device 120 manually or automatically.

Reader device 120 can algorithmically process the collected analyte data and determine whether a glucose excursion or a meal event has occurred at 306. This can occur by use of a meal event detector as described herein, which examines the analyte data for one or more analyte values that violate a threshold or other condition that is indicative of the occurrence of a glucose excursion or a meal event. This algorithmic processing can likewise be frequently repeated, e.g., each time new analyte data is received from sensor control device 102, such as in response to a user performed NFC scan of sensor control device 102 with reader device 120 or otherwise. The manual logging of meal information by the user (302) can occur contemporaneously with the monitoring (304) and processing (306) of the analyte data.

Each time new data is transferred to reader 120, the algorithmic processing can be applied to the new data, which might represent a multiple hour time period (such as the last 8 hours) to detect meal events. Steps 308-320 can be performed for each meal that is detected, starting with the most recent detected meal and repeating for every other detected meal event.

In some example embodiments, the meal event detector can use a one or more sets of predefined glucose rate of change (e.g., rise) settings to attempt to detect all meals in the analyte data. If analyte data exceeds the rate of change threshold, such as a series of data points over a predetermined time range where the average increase in value from one point to the next exceeds a threshold, then that analyte data can be characterized as a glucose excursion.

One problem that specifically arises from the use of the meal monitor application is that the meal monitor application may be either too sensitive or not sensitive enough to analyte data that suggests the occurrence of a meal event. This problem can result in the identification of too many or not enough meal events.

In some embodiments, the settings that are used by the meal event detector to determine whether a potential meal event has occurred can be adjusted. For example, each setting or threshold used by the meal event detector can be individually set by the user. In another embodiment, the meal monitor software can provide the ability to scale the sensitivity of the settings, for example by providing the option for the user to select one of a plurality of different settings each having a different magnitude.

The meal monitor software can default to a “medium” (or “normal” or “default”) mode using a group of predetermined settings or the settings input by the user. The meal monitor software can provide the option to switch to a different mode, such as a “low” mode or a “high” mode where the sensitivity or magnitude of the settings are scaled to be less likely or more likely, respectively, as compared to the “medium” mode, to consider a particular span of analyte data to qualify as a potential meal event. The “low” mode and “high” mode can each be a direct percentage scaling of the settings of the “medium” mode. For example, the “low” mode may use the settings of the “normal” mode adjusted by 5%, 10%, 15%, 20%, 25%, etc., so as to decrease the likelihood that a meal event is detected. Likewise, for example, the “high” mode may use the settings of the “normal” mode adjusted by 5%, 10%, 15%, 20%, 25%, etc., so as to increase the likelihood that a meal event is detected. While three setting options are described here any number of two or more setting options can be used. Also, the meal monitor software application can provide the option for the user to scale the sensitivity of the meal event detector in an analog or virtual-analog fashion (at least from the user's perspective), such as with a slide bar on a touchscreen.

In some embodiments, the meal monitor application is self-monitoring and optionally self-correcting, such that sensitivity of the meal event detector can be continually or repeatedly monitored against a desired baseline and, if it appears that the sensitivity of the meal event detector requires adjustment, then the user can be notified to make an adjustment or the meal monitor application can automatically adjust itself without input from the user.

FIG. 3B is a flow chart depicting an example embodiment of method 330 for automatically adjusting the sensitivity of the meal event detector. Here, method 330 can utilize a baseline integer or decimal value indicative of the normal number of meals the user consumes each day. This baseline value can be set by the user or can be a default value (e.g., one, two, three, four, five, etc.) set within the meal monitor application. Method 330 can also utilize a variable integer or decimal value that can account for typical variation in the daily number of meals that the user consumes.

In this embodiment of method 330, the meal monitor application runs the meal event detector on the analyte data from a prior time range (e.g., N days, hours, minutes) using a first set of sensitivity settings (e.g., analyte data magnitude thresholds, duration thresholds, rate of change thresholds, or others) at 332. Here, method 330 examines the analyte data over the previous N days, where N can be any desired value. Next, a determination is made at 334 whether the number of meal events detected over those N days is less than or equal to a maximum, which in this embodiment is (the baseline value added to the variation value) multiplied by N. In an example where N is three, the baseline value is three meals/day, and the variation value is one meal/day, then the determination at 334 would evaluate whether the number of meal events detected is less than or equal to twelve (e.g., the maximum).

A determination that the number of meal events is greater than the maximum indicates that the meal event detector settings are too sensitive. Thus, the routine of method 330 proceeds to 336 where the sensitivity settings are reduced. Reduction of the sensitivity settings can be to the next lowest set of predetermined sensitivity settings, or can be by a scaling factor such as by 1%, 2%, 5%, 10%, etc. Then, at 338, the meal event detector is performed on the analyte data from the prior time range with the new settings. Method 330 then proceeds to make the determination at 334 again. The process can repeat until the sensitivity settings are reduced sufficiently so as to not to violate the condition of 334.

When the condition of 334 is passed (e.g., the detected number of meal events is less than or equal to 12), the routine proceeds to make a determination at 340 as to whether the detected number of meal events is greater than or equal to a minimum, which in this embodiment is (the variation value subtracted from the baseline value) multiplied by N. In the example where N is three, the baseline value is three meals/day, and the variation value is one meal/day, the determination at 340 would evaluate whether the number of meal events detected is greater than or equal to six (e.g., the minimum).

A determination that the number of meal events is less than the minimum indicates that the meal event detector settings are not sensitive enough. Thus, the routine of method 330 proceeds to 342 where the sensitivity settings are increased. Increase to the sensitivity settings can be to the next highest set of predetermined sensitivity settings, or can be by a scaling factor such as by 1%, 2%, 5%, 10%, etc. The routine then proceeds to 338 where the meal event detector is performed on the analyte data from the prior time range with the new settings. Method 330 then proceeds to make the determination at 334 again and the process can repeat until the sensitivity settings are adjusted sufficiently so as to not to violate the conditions of 334 and 340. When the condition of 340 is met, the routine proceeds to 344 where the adjusted settings are considered to be appropriate and are used in the subsequent normal course of operation of the meal event detector.

Method 330 can be repeated at regular intervals. In one example embodiment, method 330 is performed once per day using data from the prior N days as measured from midnight to midnight to prevent the settings from changing multiple times per day.

In another embodiment, the meal monitor application can perform an automated sensitivity adjustment that analyzes the data to identify settings that are too high without regard to whether the settings are too low. For example, a self-adjustment routine can iteratively assess whether each of a plurality of settings is beneath a maximum by starting with the highest setting, determining whether the meal event detector outputs a number of detected meal events that is less than the maximum using the highest setting and if so, then using those highest settings for subsequent normal operation of the meal event detector. If the highest settings result in a number of detected meal events that exceeds the maximum, then the routine can proceed in order of decreasing magnitude through each iteratively lower setting and stopping when a setting is identified that does not exceed the maximum. That identified setting can be used for subsequent normal operation. In yet another embodiment, the meal monitor application can perform an automated sensitivity adjustment that analyzes the data to identify settings that are too low, without regard to whether the settings are too high, in a similar but reverse fashion.

Referring back to FIG. 3A, if a glucose excursion or meal event is detected at 306, then, at 308, the meal monitor application assesses whether meal information has already been entered (such as by the user at 302) that corresponds to the detected glucose excursion or meal event (collectively referred to as the “detected event”). This assessment can be performed by examining a period of time before, and optionally to a limited extent after (e.g., to compensate for inaccuracies in time keeping or entry), the times at which the detected event occurred to see if any meal information was entered during that time period. If so, then the meal monitor application can associate that meal information with the detected event at 314. If multiple meal information entries are found, then the meal monitor application can associate all with the detected event, or can associate the detected event with only the meal information which occurred closest in time.

If no meal information has been entered that can be associated with the detected event, then at 310, the user is prompted to enter the meal information. Example embodiments of user interfaces for prompting the user to log a meal or meal information are described with respect to FIGS. 4C-E. If no meal event has occurred, the user can decline or ignore the prompt. Otherwise, the meal information can be entered at 312.

Prompting can take the form of an alarm notification, such as a vibration or sound that clues the user into activating the meal monitor application to see the prompt. Alternatively, the prompt may be a notification that appears when the user next views the application.

Regardless of whether the user logs meal information at his or her own discretion (302) or in response (312) to a prompt (310), the meal information can include various levels of detail and can be entered in the same or similar fashion.

A desirable aspect of the embodiments described herein is the ease at which meal information can be entered so as to promote usage of the meal monitor application to gain a more intuitive understanding of glycemic impact of meal consumption, which in turn can improve the user's health. The meal information can be input in any desired manner, such as by the manual entry of text, by selection of the meal name from a list (e.g., a picklist or drop-down list), by selection of the meal picture from a group of pictures, by selection of recognizable indicia (e.g., a tag or code) of the meal, or any combination thereof.

The meal information can include a meal type, for example, whether the meal is breakfast, lunch, snack, dinner, or dessert, etc. The meal information can include the time range (e.g., start time and stop time) during which the meal was consumed. This can be an actual time (e.g., a start time in hour:minutes) or can be a generalized or heuristic part of the day (e.g., early morning, late afternoon, etc.), or an approximated time range (e.g., 6-7 am, 5-6 pm, etc.) with any desired level of granularity of the time range (e.g., 10 minute, 15 minute, 30 minutes, 60 minutes, etc.).

The meal information can also include the contents and portion sizes of the meal. For example, the meal may be described as including a caloric amount, protein, carbohydrates, dairy, meat, grain, vegetables, nuts, sugar, alcohol or other beverage type, etc. along with the respective amount or size for each content. This can be done on a portion by portion basis, for example, one serving of bread with one serving of peanut butter, where each portion has the respective amount of carbohydrates, sugar, protein, etc. associated with it. Alternatively, the meal may be described on solely a nutritional category basis, for example, 10 grams of carbohydrates, 5 grams of sugar, 8 grams of protein, etc. Heuristic categories (e.g., small portion, medium portion, large portion) as opposed to quantitative categories can also be used to facilitate data entry.

To further facilitate the entry of meal information, the meal monitor application can present options from which the user may select meals or information about meals. For example, meals or meal information can be presented to the user in the form of a list or array from which the user can select the most appropriate entry that describes the meal consumed by the user.

In some embodiments, the user can create a list or array of common meals consumed by the user (and/or associated information) for use by the meal monitor application, either by directly entering the list into the application, by selecting from a list of options preprogrammed into the meal monitor application, or by creating the list on a separate computing platform and uploading it to the host device on which the meal monitor application is being executed. Consumables can be added or removed at any time. As discussed herein, this data entry can be performed using a graphical user interface on the host device.

The meal monitor application can be programmed to store the information about any and all prior meals and associated meal information previously consumed by the user. That information can be presented to the user as options from which the user can select to identify a particular meal or aspect thereof that was more recently consumed. For example, when the user is prompted to enter meal information, the meal monitor application can present a list of options from which the user can choose. This list can be ordered such that the most commonly consumed or selected meals are presented first or at the top of the list, with remaining meals presented in order of decreasing frequency of consumption (e.g., the most commonly consumed meal is presented first, the second most commonly consumed meal is presented second, and so forth with the least commonly consumed meal presented at the end).

The user may often need to tailor a selected meal based on the occasion. Thus, for any meal selected, the user is given the option to customize an aspect of that meal, such as by the addition, removal, or substitution of a particular side (e.g., substitution of broccoli for peas) or drink, modification to a portion size, modification to a caloric or carbohydrate content, and so forth. In any and all instances where the user enters information about a selected meal, a similar approach can be taken where the user is provided with a list of options from which to choose, the options being presentable in order of most relevant to least relevant. For example, if a particular type of food is selected, previously consumed portion sizes of that particular type of food can be presented to the user. Again, this can be done in descending order based on frequency of consumption. For example, if chicken breast is selected as part of a meal, then based on the user's prior history stored on the device, the meal monitor application may present portion sizes to choose from in order of descending frequency of consumption (e.g., 8 ounces, 10 ounces, 6 ounces). Or the list may be presented in increasing (least to most) or descending (most to least) order of portion size. The same applies, for example, with respect to carbohydrate amount, sugar amount, protein amount, and any other aspect of meal information described herein.

Thus, selection of a particular meal can be followed by entry of information about each type of food and drink within that meal where the user's history can be used to present the most relevant options first. Of course, if no customization is required, the user can end the data entry process after initial selection of the meal itself without further specifying variations in portion sizes etc.

The meal monitor application can be programmed to filter the presentation of options based on the time of day. For example, if the user is prompted to enter information about a meal consumed during morning hours, the meal monitor application can present a list of the commonly consumed breakfast meals and/or morning snacks, etc. (in order of descending frequency of consumption) such that the user is provided only the most relevant options from which to choose (e.g., dinners are excluded). Similarly, when entering information about meals consumed in the middle of the day, only the most commonly consumed lunches and/or afternoon snacks can be presented and, when entering information about meals consumed late in the day or in the early evening, only the most commonly consumed dinners, desserts, and/or evening stacks can be presented.

The meal monitor application can also be programmed to filter the presentation of options based on the characteristics of the detected event (e.g., the magnitude and/or duration of glucose excursion or meal event) and/or the correlation between the characteristics of the detected event and prior meals associated with detected events having similar characteristics. For example, the meal monitor application can have local access (e.g., stored in local memory) or remote access (e.g., by downloaded from a server) to the user's historical glucose levels over the previous amount of time, e.g., days, weeks, months, etc., as well as meal information entered into system 100 over that same or similar amount of time.

When a meal event is detected, the meal monitor application can identify prior detected events with the same or similar characteristics in the analyte data. This can be done by reference to a database of correlations stored locally or remotely to the meal monitor application within system 100. When the user is prompted to enter meal information based on a detected event, the meal monitor application can present meal options for selection based on the correlation between the analyte data of the recently detected event and analyte data of previously detected events that already have meal information associated therewith. Time of day information can also be used in the correlation. For example, if a hypoglycemic event is detected in the early evening, and previously detected hypoglycemic events occurring in the early evening were previously associated with a particular high carbohydrate meal (e.g., a spaghetti dinner), then the user may be presented with that particular high carbohydrate meal as the first option from which to select, followed by other options in order of decreasing potential relevance.

In other embodiments, when a meal event is detected, a list of all previous meals can be displayed to the user (or accessed by a virtual selectable button labelled, for instance, “previous meals”). The user can be permitted to select one meal as corresponding to the detected meal event. The list of all previous meals can be sortable by frequency of past selections of that meal and/or by magnitude or severity of the glycemic response to that meal, which can be an average or median value if that meal has been consumed multiple times in the past. The list may be ordered any number of ways, but the preferred embodiment is to order first by the most frequently selected meals, and next by the most recent meals. Each meal can be associated with a unique identifying code. Meal names that are entered by the user with text that is not identical but that resemble each other can be assigned the same code. If the same meal is entered and selected one or more times, that unique identifying code will be stored multiple times associated with the different times for this same meal. When displayed on a list sorted by glucose response magnitude, the meal monitor application can detect when meals are repeated by detecting that they have the same unique code. The meal monitor application can create a list of all the glucose responses for each repeated meal and determine the mean or median of these responses and associate this with the unique code. When this is done for all of the unique codes, then the final list for display can be generated for all of the unique meals including meals entered only once, and repeated meals, and the list can be sorted by the glucose response magnitude associated with each unique code.

If the user selects a meal event that is associated with an undesirable glycemic response, then a real-time notification can be generated and output to the user to warn that user of the potential glycemic impact of the meal that was just consumed.

Such real time feedback (i.e., feedback returned immediately from the user's perspective), can help the user internalize the undesirable effect of the consumed food. For example, if the type of meal event detected is a dinner meal, then the real-time notification can display the average glycemic response of all other meals of that type (dinner) along with the average glycemic response of the type of meal that was just consumed and an indication of the difference (e.g., the recently consumed meal has a typical glycemic response 20% higher than the average glycemic response of all other dinner meals). The real-time notification can also warn the user to eat less or take an offsetting medication such as insulin.

In one example embodiment, the notification can include a graphical display of the user's current analyte data over time, with an average of all previous glycemic responses to that meal type superimposed on the current analyte data graph synchronized to the meal start time. One such example is depicted in FIG. 3C, where a trace 343 of the user's current analyte data is displayed over time. The meal start time is indicated at 344 and the last collected analyte measurement is indicated at 345. Upon selection of a meal type that is associated with one or more undesirable prior glycemic responses, the graph of FIG. 3C can be displayed with an overlaid trace 346 representing the average glycemic response in the user for all prior consumptions of the same meal type (or all previous times in which an undesirable excursion occurred with that meal type). The start of the overlaid trace 346 can be aligned with the analyte data at the meal start time 344. Thus, the user is given real-time visual feedback of the likely result of consumption of that problematic food. Alternatively, multiple traces can be overlaid where each trace represents a single past excursion from consumption of that meal type.

Alternative outputs include reports that are available from a menu provided by the meal monitor application. Examples of reports include a list of detrimental meals (e.g., a “bad meal” list) and a list of beneficial meals (e.g., a “good meal” list). The bad meal list can be a list of previous meals sorted by descending glucose response magnitude, whereas the good meal list can be of previous meals sorted by ascending glucose response. The lists may be further modified by only including meals, for instance on the bad list, that exceed a predetermined or user defined threshold of glucose response magnitude. For the good list, only meals that are less than a predetermined or user defined threshold of glucose response magnitude can be included. Also, for the list of previous meals used to select from when entering meal information, symbols may be used to identify meals as good, bad or neither, as described above. Good and bad meals may be identified in other ways for this selection list, such as inclusion in respective sub-lists.

Also, reports and periodic notifications may be provided that keep a running total of the quantity of good meals and bad meals over an elapsed period of time, such as one week or two weeks. For instance, each time the user selects this report, the meal monitor application can count the number of good meals and the number of bad meals and display the result. In addition, the application can provide a user interface that allows the user or health care provider to set a goal for the maximum number of bad meals in a period of time. The report display can show the current number of bad meals next to the goal. Alternatively, the application may generate periodic notifications regarding these metrics, where the period is, for instance, weekly.

The application can process new data (whenever available either automatically or when manually queried) to detect meals, measure glucose responses to detected meals, and perform the running calculation on the meals. Alternatively, the application can determine glucose responses whenever they are needed for analysis or reporting; that is, glucose responses are retrieved from memory but recalculated when needed. When the running calculation of, for instance, bad meals per week exceeds a threshold (or falls below a threshold), a notification can be generated indicating to the user that their eating habits have digressed (or congratulating that the goal has been met). Notifications may take a form similar to that of a standard lock screen notification on an Android or iOS phone, or they may be presented on specific display areas on common application screens such as the home screen or the glucose result screen.

The user has the option to input a photograph or image of the meal. This image may be entered to supplement other information also entered to describe the meal. Alternatively, this image can constitute the entire description of the meal and its contents. This streamlined data entry can greatly facilitate the ease at which the user enters data in logging a meal or responding to a prompt, thereby encouraging use of the meal monitor application. If an image is entered, that image can be presented along with or instead of a textual description of the meal and/or contents thereof, such that the user need only recognize the image in a list of options presented to the user for data entry. Thus, when the user is presented with options for identifying a meal in response to the detection of an event, those options can be presented solely in the form of text, solely in the form of icons, or solely in the form of images, or those options can be presented in a combination of text, icons, and/or images.

In some embodiments, the imagery of the meal can be analyzed using image recognition techniques to identify attributes of the meal such as recognizable components of the meal. The components of the meal may be recognized using meal component recognition algorithms based on spectral boundaries defined within the image. If recognized, the meal imagery can be given a name by the meal monitor application that describes the meal contents, e.g., a picture of chicken and broccoli can be labeled as “chicken and broccoli.” The meal monitor application can then provide options to the user to specify further information, e.g., the portion amount, nutritional content, etc., for the particular type of meal detected from the imagery. That meal can also be linked to prior instances where the same type of meal occurred in the user's meal history and used in correlating detected events to particular types of meals.

FIGS. 4A-E depict example embodiments of graphical user interface (GUI) visual arrangements or screens. These screens can be displayed on any of the embodiments of reader device 120, drug delivery device 160, or personal computer system 170 described herein.

FIG. 4A depicts an example embodiment of a screen 402 for logging meals and associated meal information, for example, as would be used at 302 of method 300 (see FIG. 3A). Screen 402 can include indications 404 and 406 of analyte levels determined from data received from analyte sensor 104 (not shown). Indication 404 is a numerical display of the user's current or last received analyte level (e.g. mg/dL). Indication 406 is a graphical display depicting a trace or curve of the user's analyte level (y axis) over time (x axis) against a shaded region 407 indicative of normal or desired analyte level range. Such an indication 406 can show data over any desired time period (e.g., eight hours or other periods).

Screen 402 can include visual indicators for indicating various attributes of the data. For example, graphical display 406 can include a highlighted or otherwise accentuated indicator or marker 408 of the data. Here, indicator 408 is indicative of a glucose rate of change that exceeds a desired maximum rate of change, e.g., a rapid rise (or in other instances a rapid fall) of the analyte data over time. Specifically, indicator 408 is a highlighted area beneath the analyte level curve corresponding in time to the occurrence of the rapid rise. Thus, an observer can more readily appreciate whether recent events or excursions have occurred, as well as their nature.

Screen 402 can be a home screen for the meal monitor application or can be accessed from a home screen or other higher level page. Here, screen 402 is accessed from a home screen and includes a selectable home screen back button 410 to return thereto, which can likewise redirect back to any other higher level page.

Screen 402 also includes a Log Meal selectable button or field 412. Selection of the Log Meal button 412 can direct the user to screen 420 depicted in FIG. 4B. If the device on which the meal monitor application is operating includes a camera, then screen 420 can include a selectable Take or Select Photo button or field 422 that, upon selection, can open a camera application with access to the device's camera to capture a photograph or image of the meal for storage and association therewith. Selection of button 422 can also give the user the option to select a photo from a gallery of photos previously used to log meals, or from a gallery of photos stored in the device's general purpose photo gallery. If the user captures or otherwise selects a photo of the meal, then that photo can then be displayed on screen 420. In some embodiments, selection of Log Meal button 412 of FIG. 4A can cause the meal monitor application to immediately open the device camera application to allow the user to take a photo and thereby further streamline the image entry process.

Also included on screen 420 is a textual entry input location 424 for the user to enter free text information describing the meal. Selection of this location 424 opens a text editor which can be used to enter textual information about the meal. This information, along with the photograph, can be associated with the meal and can form part of a data structure representing the meal. Although not shown here, a pick list or drop-down list can also be included on screen 420 that the user can select to choose from a previously stored list of options for the meal or the meal's information, as described in detail herein.

Although not all variations are depicted, it is recognized that all aspects of meal information described herein can be input via one or more screens similar to those described with respect to FIGS. 4A and 4B, including, but not limited to, the entry of time of meal information, meal portion information, nutritional content, representative meal icons, and so forth.

After the meal information is entered, either with a photo, textual description, or selection of a predetermined option, the user can select a Save button or field 426 to save the data to memory, as a data structure associated with and digitally describing the meal.

FIGS. 4C-E depict example embodiments of screens 428 and 430 for prompting a user to enter meal and associated meal information, for example, as would be used at 310 of method 300 (see FIG. 3A). Like screen 402 of FIG. 4A, screens 428 and 430 can include indications 404 and 406 of analyte levels determined from data received from analyte sensor 104 (not shown). Also like screen 402, indicator 408 is indicative of a glucose rate of change that exceeds a desired maximum rate of change, e.g., a rapid rise (or in other instances a rapid fall) of the analyte data over time. Specifically, indicator 408 is a highlighted area beneath the analyte level curve corresponding in time to the occurrence of the rapid rise.

This rapid rise can be the glucose excursion or meal event detected at 306 of method 300 (see FIG. 3A), which is the impetus to prompt the user at 310 to enter meal information. Screen 428 of FIG. 4C is an example of a home screen of the meal monitor application. Here, when an excursion or event is detected the home screen displays a pop-up window 429 that can overlay the home screen and notify the user that one or more possible meals were detected and optionally give the time when each of those meals were detected. Pop-up window 429 includes a selectable field where the user can choose to log a meal for each time period corresponding to the detected excursion or event, and can also include a selectable field where the user can choose to ignore the notification of window 429. Here, screen 428 includes a historical graphical display 406 of the user's analyte level with indicators 408 for each detected glucose excursion or meal event. When pop-up screen 429 is displayed, the corresponding detected events where meal information has not yet been entered can be graphically identified or distinguished from those events where meal information has already been entered or where it is not required.

In an alternative embodiment, upon detection of a glucose excursion or meal event, the meal monitor application can initiate or display screen 430 of FIG. 4D to identify the detected event to the user (at least in part by way of indicator 408) and prompt the user to enter a meal information by selectable Event Detected button or field 412. Alternatively, the meal monitor application can display the Event Detected button 412 on another screen currently being displayed to the user. The Event Detected button 412 can be alternatively be labeled as a Meal Detected button.

Selection of the option to log the meal in pop up screen 429 or selection of the Event Detected button 412 can cause the meal monitor application to transition to screen 420 of FIG. 4B, reproduced again as FIG. 4E for ease of illustration. There, as in the example embodiments described with respect to FIGS. 4A and 4B, the user can enter meal information photographically via button 422 and/or textually via free text field 424, and save that information via button 426. Because detection of the meal event occurs after consumption of the meal, the user may choose to photograph packaging for the meal, or scan a barcode of the meal packaging, instead of photographing the meal itself. All variations to the meal information entry process described with respect to FIGS. 4A and 4B (including, but not limited to the pick list and drop-down list variations), including all variations to the meal information entry process described herein but not shown in FIGS. 4A and 4B can likewise be applied here.

When a user logs a meal at his or her own discretion, for example using screen 420 of FIG. 4B, the meal monitor application can require the user to describe the time at which the meal occurred in any of the formats described herein. When the meal monitor application detects an event, then the application already has access to time information about the event. Thus, when the user is prompted to enter information about the meal event such as, for example, using screen 430 to access screen 420, that determined time information can be displayed to the user so that the user has an option to edit the time information if not accurate. If a rise in the analyte data is used to detect a meal event, then the time at which the rise episode starts can be the default displayed meal time, with compensation for sensor and digestive-based lags as described herein.

In some example embodiments, the data entry process is streamlined to further minimize the burden on the user when inputting data. The lower the burden the more likely the user is to utilize the meal monitor application, and thus enjoy its benefits. In one example embodiment, the meal monitor application can be configured so that it does not actively prompt the user for any nutritional information about the meal nor the meal type (e.g., breakfast, lunch, or dinner) when accepting a log entry from the user (e.g., step 302 of method 300) and/or when accepting information from the user in response to a prompt for meal information (e.g., step 310 of method 300). In another example embodiment, the meal monitor application is configured so that it only accepts an image of the meal and/or a free textual description of the meal and does not permit any other information about the meal to be entered when accepting a log entry from the user and/or when accepting information from the user in response to the prompt.

Referring back to FIG. 3A, once the information for a meal event has been entered at 312, the meal monitor application will associate the analyte data with that meal information in memory at 314. Specifically, the analyte data occurring around the time of the meal event can be associated with the information for that meal event. The analyte data selected to be associated with the meal event can be chosen based on the time during which the meal event occurred and optionally a period of time after which the meal event occurred to reflect changes in the analyte level due to digestion of the meal.

In some embodiments, analyte data occurring from the time at which the meal began until a period of time after the meal ceased can be associated with the meal event. Also, analyte data occurring from the time at which the meal began until the conclusion of a detected glucose excursion can be associated with the meal event. In some embodiments, analyte data collected during a fixed range of time around the meal event is associated with the meal event, for example, any combination of from one, two, or three hours before the meal event (e.g., as measured by initiation of the meal event, a median time of the meal event, or a conclusion of the meal event) until one, two, three, four, five, six, seven or eight hours after the meal event. In each case, times over which the meal event occurred can be identified based on information entered by the user or can be identified algorithmically through analysis of the analyte data.

Data collected from analyte sensor 104 may temporally lag the user's actual blood glucose level, for example if sensor 104 is sensing data from within the user's interstitial fluid, and to a lesser degree, dermal fluid. Thus, correlation of analyte data to meal consumption times should take into account this sensor-based lag, which may typically be on the order of 3 to 20 minutes depending on fluid type and sensor placement. This lag is in addition to lag time that results from absorption of food through the digestive process. In other words, there is a time delay between the time when food is consumed and the time when the results of that consumption are reflected in the user's blood glucose level. Thus, in some embodiments, the analyte data associated with the meal event is selected as described above with additional compensation for digestive lag and/or sensor-based lag. For example, the first analyte data points to be associated with a meal event may be based on the commencement of that meal event, but delayed by 10 minutes (five minutes to compensate for digestive lag plus five minutes to compensate for sensor-based lag).

The association of analyte data with a meal event can be used in determining a glycemic impact of the meal event at 316. In some embodiments, determination of the glycemic impact of the meal event can be done algorithmically with reference to analyte data contemporaneous with the meal event, and this algorithmic processing can constitute both steps 314 and 316. The determined glycemic impact can be determined quantitatively in terms of maximum (peak) or minimum glucose level, median or mean glucose level, minimum-to-maximum change in glucose level (delta (A) glucose value), percent variability of glucose level, duration of glucose response, rate of change of glucose level, area of the glucose response, and any combination thereof.

The meal event detector outputs information about the glycemic response to the meal that can be used to characterize the magnitude or severity of the response. For example, the meal event detector can output the start time and the peak time for each detected meal event. The meal-start glucose can be the glucose value at the meal event start time and the peak glucose can be the glucose value when a rise episode of the detected meal event peaks. The difference in these glucose values can be determined to provide a delta glucose measure of the glycemic response to the meal.

In other embodiments, the “area” of the glycemic response is determined in terms of glucose and time; for instance, a sum or integration of each glucose reading is determined from the meal start time to either a) the next meal start time, or b) when the glucose readings fall within a threshold value of the meal start glucose value (e.g., within 10 mg/dL), as it can be difficult to accurately assess where the glycemic response ends. This sum or integration value is proportional to the glucose multiplied by time area of the glucose response. Each of the metrics described herein, as well as others, can be used as the basis upon which to rank and/or sort the glycemic response to meals output to the user as described below.

Another problem that specifically arises from the use of a meal monitoring and detection software is that in some instances multiple meal events may be detected and/or logged in close temporal proximity to each other. This problem can result in the identification of too many meal events when, from the user's perspective, the events were part of the same meal.

In these cases, the meal monitor application can cluster or group the meal events into one meal cluster. For example, meal events logged or detected within a time range condition (e.g., one hour, 90 minutes, etc.) of each other can be merged into one meal cluster. The meal monitor application can then analyze and output glycemic response data to the user describing the meal cluster as a whole as opposed to each separate event therein, which is intended to correspond to the user's concept of a meal (e.g., dinner may be one cluster of different courses). Thus, each meal event or meal cluster that is analyzed and displayed to the user is separated by at least the value of the time range condition, which can result in a more natural information output that is easier to comprehend. To facilitate this, the meal monitor application can avoid placing a restriction on the number of meal events considered per day.

FIG. 3D is an annotated graph 350 of analyte data over time displaying an example set of data where four different meal events (352-1, 352-1, 352-3, and 352-4) have been automatically detected or manually logged. These four different meal events are considered a single meal cluster 354 because each event 352 is within a time range condition, which can be a predetermined factory-set condition or a condition set by the user. In this example, the time range condition is one hour, and all four events 352 are grouped together because each individual event 352 is within one hour of at least one other event 352.

In some embodiments, a maximum time limit can be placed on the length of time from a first event 352 that the meal monitor application will consider other events for clustering. For example, the time range condition can be one hour with a meal cluster maximum length (not shown) of 90 minutes after the first event 352-1, in which case meal events 352-1, 352-2, and 352-3 would be grouped as one meal cluster and meal event 352-4 would be considered a separate meal event from the cluster.

As described herein, the time of the meal event can be the time input by the user in the manual logging process, the time at which an analyte episode corresponding to the meal event is first detected (e.g., the start of a rise), the time at which an analyte episode corresponding to the meal event is first detected plus the addition of a delay (e.g., 15 minutes, 25 minutes), the mean or median time determined from the duration of the analyte episode (e.g., the meantime from start to end of a rise or the time of the median data measurement collected during the rise), or any other desired time.

In some embodiments, multiple different meal events detectors, based upon different algorithms, can be used. Each individual meal event detector routine can be independently run on the collected analyte data to identify detected meal events and/or their start times. The meal monitor application can then choose which results to use for analysis and display to the user. For example, if each meal event detector outputs a different start time for the same meal event, then the meal monitor application can average the two and use the averaged meal start time for analysis and display. In another example, the meal monitor application may consider an event to be detected only if each meal event detector actually detects it. In yet another example, the meal monitor application may consider an event to be detected if at least one meal event detector actually detects it.

In the example depicted in FIG. 3D, a pre-meal or meal-start glucose value 356 can be the glucose value at the start of the first meal event 352-1 of meal cluster 354. If necessary, this value can be obtained from the data measurement occurring closest in time before or after the time of meal event 352-1. If no data measurement is present within a maximum time range, such as 30 minutes, then the meal monitor application may consider the pre-meal glucose value to be unknown.

A meal peak glucose value can be the highest glucose value occurring in the analyte data collected during or after meal cluster 354. Meal peak glucose time range 358 denotes an example time range window that is searched for the occurrence of the highest glucose value. Time range 358 can start it the time of the first meal event 352-1 and can end at the time of the last meal event 352-4, or more likely, can and the predetermined time after the last meal event 352-4. In the embodiment depicted in FIG. 3D, time range 358 begins at the time of the first meal event 352-1 and ends two hours after the last meal event 352-4. Thus, the meal peak glucose value would be the magnitude of values in region 360 where the analyte data peaks and maintains a constant level. If no readings are present in window 358, the meal peak glucose value can be considered as unknown.

A meal delta glucose value can be determined by subtracting the pre-meal glucose value from the meal peak glucose value. If one of or both of those values are unknown, then the meal delta glucose value can be considered as unknown.

Referring back to FIG. 3A, the determined glycemic response or impact can then be output to the user at 318, such as visually on a display of the smartphone, as will be described below with respect to FIGS. 5A-F. In many embodiments, this is done immediately with a presentation of information about the corresponding meal event so that the user can immediately understand the impact of a particular meal on the user's analyte levels, making the application compelling to use. The determined glycemic response can be output in quantitative and/or qualitative terms. For example, the determined glycemic response can be output in the same quantitative measure in which it was determined at 316. This can be done in text and/or graphical form. Also, the determined glycemic response can be output qualitatively, for example, described in text as low or minor, moderate or medium, or high or severe, or any synonym thereof. Less or more graduations of magnitude can be used as well. The determined glycemic response can also or alternatively be output as an icon or other imagery (e.g., colored shapes of various degrees of magnitude).

System 100 can also (or alternatively) issue a medication or other treatment output at 320. In certain embodiments the output can inform an individual, such as the user or a medical professional, that medication (e.g., insulin) or treatment may be warranted, for example, due to the risk or occurrence of a high glucose excursion or hyperglycemia. This notification can be a visual and/or audible notification output by reader device 120.

An output issued at 320 can supply information directly to the user's medication or treatment program on the same device or a different device in communication with the device executing the meal monitor application. This information can be used by the treatment program in determining whether a modification to a user's treatment profile (e.g., a basal insulin delivery schedule or a bolus dose) is warranted or to prompt the user as to whether a change to the user's treatment profile should be implemented.

An output issued at 320 can cause the user's drug delivery device 160 to administer medication to treaty a potential or actual high glucose condition, such as by administration of a bolus dose or by a modification to a basal dosage profile or schedule. This can be done with the user's approval, such as in a semi-closed loop, or automatically without the user's approval, such as with a true closed loop or artificial pancreas.

The meal monitor application can also issue any of the aforementioned medication or treatment outputs when a user indicates that a meal is or was recently consumed where that meal has previously caused a glucose excursion, such as a rapid rise or high glucose level. In this manner system 100 can pro-actively treat the anticipated glucose change without waiting for the detection of the actual change with sensor 104.

Outputting information from the meal monitor application to a medication or treatment program and/or drug delivery device enhances the ability of the program or device to implement accurate therapy for the user. More accurate information about the user's meal history is captured and made available to the program or device to make the appropriate, sometimes subtle, adjustments to the user's treatment. This can, in turn, reduce the occurrence of errors in medication administration (e.g., over or under-dosing), which constitutes an improvement to the functioning of electronic and mechanical systems with drug delivery capability.

FIGS. 5A-F depict example embodiments of GUI screens that can display output information to the user or any other individual in step 318 of method 300 (see FIG. 3A) or at other times during use of the meal monitor application. These screens can be displayed on any of the embodiments of reader device 120, drug delivery device 160, or personal computer system 170 described herein.

FIG. 5A depicts screen 502, which includes a series, listing, or group 500 of logged meals 505-1 through 505-N. Group 500 is generally referred to herein as a ranking report 500. Ranking report 500 shows all or a subset of meal events 505 (and activity, if applicable) for which data has been collected. If a meal cluster was identified, then the meal events within the cluster are grouped together and displayed as one meal event 505. The ranking of events 505 can be ordered by the magnitude of the glycemic response. When presented to the user, ranking report 500 can display starting at the top of the list, showing the meals 505 that resulted in the largest glycemic responses first, followed by those that resulted in glycemic responses of decreasing magnitude.

In another embodiment, ranking report 500 can display the most recent meal causing a glucose excursion and the user can scroll (upwards or downwards) through the list which is again sorted in order of highest to lowest glucose excursion magnitudes. In yet another embodiment, ranking report 500 can display the meals causing glucose excursions in order of most recent to least recent. Toggle sort buttons can be provided that allow the user to change the criteria upon which the ranking in report 500 is determined (e.g., change ranking by meal type, date and time, glucose excursion magnitude, and the like).

In the embodiment depicted in FIG. 5A, for each meal 505 within ranking report 500, an image of the meal can be displayed if available, along with the date and time at which the meal was consumed, along with an indication of the magnitude of glucose response, which can be presented in any of the output forms described herein. Instead of or in addition to an image, the name of each meal 505 can be described textually. In addition to ranking meals by their glycemic response, ranking report 500 can rank occurrences of a single particular meal type by their glycemic response. For example, portion sizes, times of day, and glycemic response for a commonly consumed spaghetti dinner can be ranked and displayed.

If a glucose excursion occurred but no meal information was entered, then the time, date, and magnitude of the glucose excursion can be displayed in report 500 along with a selectable button or field which, upon selection, will prompt the user to enter information about the meal, such as by displaying screen 420 of FIG. 4B. If there is meal information but no photo, then an missing photo indicator can be displayed so that the date and/or time is aligned with the other entries in report 500.

If meal event detection sensitivity settings or glucose excursion sensitivity settings are adjusted during the use of the meal monitor application, then the data displayed in ranking report 500 can be retroactively adjusted so that all data is displayed with one common array of sensitivity settings. Thus, each time the user displays a ranking report 500 or otherwise displays historical meal or analyte information, the meal monitor application can recalculate the displayed information to retroactively compensate for sensitivity adjustments.

Selecting or clicking on a meal in ranking report 500 can initiate a screen with additional information about that particular meal. FIG. 5B depicts an example embodiment of such a screen 512 that can include a graphical display 514 of historic glucose (e.g., 8 hours, 24 hours, 48 hours, or other) surrounding the meal event. Graphical display 514 can include any increment of time prior to, after, and surrounding the meal. In one example embodiment, graphical display 514 is an eight hour period of time with two hours of data prior to the start of the meal event and six hours of data after the start of the meal event. Graphical display 514 can also include time periods of any of the various ranges of analyte data associated with the meal event at 314 of method 300 (see the description with respect to FIG. 3A).

An excursion indicator 408, in this case a shaded area under a glucose curve, can be displayed for each excursion detected in the analyte data of graphical display 514. The occurrence of the selected meal event along with every other meal event within the period of time of graphical display 514 can be indicated by a food icon 516, a photo of the meal, and/or text describing the meal. Alternatively, meal events that are clustered together can be indicated as only one event. Selection of a particular meal event can cause another screen 512 to display, which is instead focused on that newly selected meal event. Alternatively, selection of a meal event can cause a pop up window to display with information about that particular meal event.

In another embodiment, selecting an individual meal from screen 502 of FIG. 5A can display an embodiment of screen 512 that includes graphical display 514 along with an image of the selected meal, with a summary of the meal contents of that meal (e.g. portion type, portion size, etc.) and/or any free text entered by the user with respect to that meal. Thus, all or most of the information entered by the user to describe the meal is displayed, along with historical analyte data around that meal event.

In another embodiment, each meal displayed either in the ranking report or elsewhere may be displayed with the associated glucose trace. When displayed with other meals on the ranking report, the user can compare features of different meals, such as the relative peak glucose time and how long the glucose response lasts. The display may highlight some of these features, such as displaying the numeric elapsed time of the peak glucose relative to the start of the meal.

FIG. 5C depicts an example embodiment of a screen 520 showing an organizational layout of information for display to the user about a particular meal, for example such as would be displayed upon selecting a particular meal from ranking report 500. The date where a time corresponding to the meal event first occurs is displayed in region 522, followed by the time (or time range if supplied) of the meal event. Beneath one or more images 524 of the meal can be displayed, for example, if the user captured a photo of each course of the meal. Meal peak and meal delta glucose values can be displayed to the right at 526, along with any free textual description of the meal at 528.

FIG. 5D depicts an example embodiment of a screen 530 populated with example information about a particular meal. Screen 530 is similar to screen 520 but also includes indications of detected meal events 532-1 and 532-2 for which no information is yet been entered, e.g., a missed entry. Each meal event 532 is followed by a user selectable log buttons 533-1 and 533-2 for logging information about the meal event and user selectable ignore buttons 534-1 and 534-2 for selection if the user chooses not to enter information about the meal or if no meal in fact occurred. If the user chooses to log information about the meal, then a log information screen such as screen 420 of FIG. 4E can be displayed with the meal time pre-populated as the time of the missed entry. If the user returns to screen 530, the information should be recalculated to account for the newly logged data. If the user chooses to ignore the missed entry, then a log of missed entries can be updated, and that ignored entry can be removed from screen 530.

FIG. 5E depicts an example embodiment of a screen 540 similar to screens 520 and 530. Here, screen 540 includes six photographs of a meal cluster occurring between 10:50 am and 11:20 am. In any of the embodiments described herein, selection of a particular photograph can cause the display of a pop up window containing a larger display of that image.

FIG. 5F depicts an example embodiment of screen 550 similar to the previous screens but with an indication of logged or detected exercise or other activity 552 and the times at which that activity occurred in relation to the meal event described in screen 550.

Referring back to FIG. 3A, method 300 can repeat itself such that the meal monitor application continues to receive, monitor and store the analyte data (e.g., step 304 of method 300) and search for detected events 306 indefinitely. The recently entered information about a detected or logged meal can be included in the options presented to the user for entering meal information each time a new event is detected.

The meal monitor application can be used with software that monitors and collects information about the user's activity level. Thus, for each of the embodiments described herein that relate to the logging of meals, prompting for information about meals, associating analyte data with meals, determining glycemic impact of meals, and/or outputting information about the relationship between a meal and the user's glucose level, it should be understood that those embodiments can likewise be applied to an activity performed by the user that is not the consumption of the meal. Common examples of such activities are exercise, work, and rest. All such activity related embodiments are within the scope of this disclosure. To assist in the collection of activity data, each sensor control device 102 can include or otherwise be configured to operate with an activity monitor that continually or repeatedly measures the level of activity for each wearer (e.g., calories burned per time of day) and/or a heart rate monitor that monitors and enables the recording, by system 100, of the wearer's heart rate. Additional example embodiments of analyte monitoring systems that include or operate with activity monitors and heart rate monitors are described in the U.S. Provisional patent application titled “System, Device and Method of Dynamic Glucose Profile Response to Physiological Parameters,” filed on the same date as the present application, and attached hereto as appendix A. The embodiments described in appendix A can each be used with any and all embodiments described herein for any and all purposes.

The information output by the meal monitor application, such as ranking report 500, provides the user with concrete and immediate information regarding the impact of their meals on their glucose levels. This output information can help users learn to avoid or minimize certain meals in their diet that they did not realize were impacting their glucose levels, e.g., that they did not realize were driving their glucose levels so high. This output information can also help users learn to control the portion sizes of their meals by seeing the relative impact of different portion sizes on their glucose levels.

The meal monitor application can also encourage the individual to experiment with different meals, times, components, and can be compelling and fun to use. This meal monitor application promotes the use of analyte sensors by a large demographic including people with Type-Two diabetes, pre-diabetes, metabolic syndrome and people without diabetes—basically any person who is motivated to improve their health by changing their diet and/or activity habits.

The meal and/or activity monitoring applications described herein can have substantial benefits for individuals that are newly diagnosed with Type-Two diabetes or pre-diabetes. Often, when an individual is confronted with starting diabetes medication, they are highly motivated to try diet and exercise modifications in order to avoid or delay the need for medication.

Thus, a clinical scenario for use of various systems and software disclosed herein is for individuals that are newly diagnosed, for example, based on a fasting glucose test and/or an A1c test, to follow up by wearing a sensor control device 102 that interfaces with a reader device 120 that can be blinded so that the user's current and historical analyte levels are stored but not shown to the user. The user wears sensor control device 102 for a period of time, e.g. two weeks, after which the analyte data is collected by a medical professional and the analyte patterns are revealed to the user. This blinded usage minimizes the impact of the system on the user's habits and tendencies, and thus gives the medical professional a better understanding of the specific glycemic issues and how best to address them with medication. At this point the physician can give the individual the option to try to address their diabetic condition with diet and exercise using the software applications described herein, in conjunction with nutritional training and an exercise plan. This may be followed up with another sensor control device 102 and reader device 120 to allow the medical professional to evaluate progress, and to determine if medication or further diet/exercise intervention is still required.

Additional metrics and reports geared towards the medical professional can be included. For example, a study on 14 normal glucose tolerance (NGT), 12 impaired glucose tolerance (IGT), and 16 non-insulin dependent diabetes mellitus (NIDDM) individuals showed that the first phase insulin response to an intravenous glucose challenge is absent in Type-Two diabetes mellitus (T2DM) subjects. With sufficient monitoring of meal information paired with glucose excursion tracking, changes in the first phase insulin response can be tracked by the medical professional.

Moreover, a quarter of the world's adults have metabolic syndrome. These adults have a five-fold higher risk of developing Type-Two diabetes. In addition, evidence indicates that most of the damage to a person's beta cells is done prior to pre-diabetes diagnosis, demonstrating the need for intervention prior to diagnosis. Since progression to Type-Two diabetes starts from impaired glucose tolerance (IGT) well before impaired fasting glucose (IFG), sensor-based hyperglycemia and post meal excursion based metrics that have been shown to reliably predict the onset of or diagnose Type-One diabetes may be adaptable for those with metabolic syndrome. Thus, the software applications and systems described herein for newly diagnosed diabetes individuals is therefore applicable to the metabolic syndrome population.

Furthermore, the software applications and systems described herein have applicability to those wishing to lose weight, as a general correlation between glucose excursions and weight gain is known.

### Embodiments of Experiential Medication Dosage Calculations

Estimating quantity of carbohydrates in a given meal is difficult, particularly for home cooked meals where the recipes have not been analyzed for protein, fat, and carbohydrate content, and where home cooks may have their own flair when preparing a given meal. However, a home cook generally prepares the same meal in the same way each time. Likewise, restaurant meals may not have content (e.g., protein, fat, carbohydrate. etc.) analysis, but repeat themselves in preparation and portion. In addition, whereas people may have a wide meal palate, on a routine basis, most meals consumed by a given person are relatively limited in choice and repeat over a monthly or weekly basis. For example, consider a typical person who eats lunch in the workplace cafeteria where the menu typically rotates among a limited number of choices, and hence, this person eats the same lunch many times over a year.

As such, it is feasible to empirically observe a patient's average analytic (e.g., glucose) response to a given meal. The empirically observed glucose response may be used to estimate insulin bolus for a given meal. In addition, the patient's empirically determined glucose response to a given meal may also be used to augment their diabetes management by steering them away from certain meals and towards others which permit better glucose management.

Described herein are a number of example embodiments of systems, devices, and methods that collect information about a diabetic user's meals and the analytic (e.g., glucose) response to those meals, associate medication dosage information with those meals, and then utilize the recurring nature of those meals to provide the user with a quick and efficient interface to determine a medication dosage and/or modification to that dosage to compensate for the consumption of that meal.

As explained in greater detail herein, rather than estimate the carbohydrate content for each meal and manually enter this value into a bolus calculator, these example embodiments allow the user to select his or her meal from a menu interface that can then directly provide the proper bolus amount for the meal. These embodiments allow a user to associate medication dosages with meals that the user commonly consumes and thus avoid the hassle and potential risk of error involved in computing carbohydrate content for a meal based on each ingredient within that meal.

Because the embodiments can be customized to the user's typical experience they can be referred to as “experiential” tools. For ease of discussion herein, the example embodiments will be described in the context of insulin bolus dose determinations and will be generally referred to as the “experiential bolus assistant” or “EBA” for short. However, it is stressed that these example embodiments can be used with all types of insulin (e.g., long-acting insulin, intermediate, short-acting insulin, etc.) and all other types of diabetes medications other than insulin. The example embodiments can also be used to determine types of dosages other than bolus dosages, such as basal dosages or basal time-varying dosage profiles, etc.

Like the meal monitor application, the EBA described herein can be implemented by a software application that is stored on and executed by a processor-based device, such as any one of the reader devices, drug delivery devices, or other computing devices described herein. In certain embodiments, the EBA is implemented as one or more downloadable software applications (“an App”) on a reader device such as a mobile telephone or smartphone, from which the EBA can communicate with a remote server (e.g., a cloud-based server), which can provide more comprehensive and robust analytics accessible by the user on the same or a second computing device.

The same mechanisms described with respect to the meal monitor application that allow a user to define consumables (e.g., a meal, a snack, a drink, a portion thereof, or a combination thereof) in any fashion that is convenient to the user can also be utilized in or with the EBA, although it some cases it can be beneficial for the user to avoid characterizing each portion of a meal consumed but rather to describe each meal “as a whole”—in other words to describe the meal as the sum total of all consumables consumed during a single sitting (e.g., a meal time such as breakfast, lunch, or dinner).

When used with an analyte monitoring system 100, these embodiments can capture, categorize, and index glucose responses to the meals and meal-time insulin doses (administered to compensate for the meal) and thus provide the user with additional data from which the user's insulin dosages can be perfected or “fine-tuned.” In addition, over time, the example embodiments can provide recommendations as to the titration of the bolus amount for each meal.

The EBA is described herein primarily in the context of determining corrective bolus amounts to compensate for the consumption of a meal. However, these EBA embodiments can include functionality (either in addition to the meal functionality or as an alternative to it) that allows for the calculation of corrective carbohydrate or medication amounts to address the patient's response to activity such as work, exercise, and even rest. Thus, to the extent that the example embodiments described herein make reference to meals, menu items, images of meals, medication amounts and titrations to correct for meals, those references likewise apply to activities, types of activities, images of activities, medication and/or carbohydrate amounts and adjustments thereto to compensate for activities.

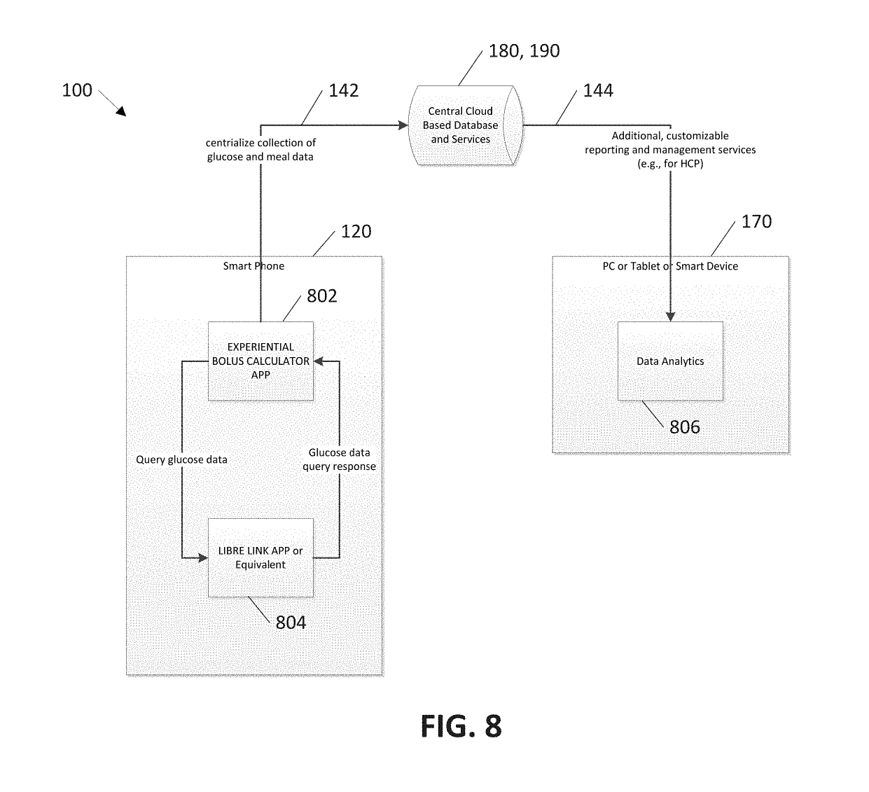

Turning now to the description of the EBA in greater detail, FIG. 8 is a block diagram depicting an example embodiment of system 100 configured to operate with the EBA in modular form. Here, the EBA (802) is in the form of a downloadable app that has been downloaded (e.g., through an “app store” or equivalent virtual environment) and installed on a smartphone reader 120. In this embodiment, a second app 804 has also been downloaded and installed on smartphone reader 120. App 804 is responsible for interfacing with sensor control device 102, processing analyte data received therefrom, and configuring that data for display to the user. App 804 can essentially convert the commercial smart phone into a dual smartphone reader device 120. An example of app 804 is the commercially available LIBRE LINK application, which is configured for operation with the FREESTYLE LIBRE analyte monitoring system provided by ABBOTT DIABETES CARE INC., although similar other applications can be used as well. While the applications 802 and 804 are described here as separate apps, they can also be combined into a single download app with a single access icon on the reader 120. This combined app can also include the functionality of all of the embodiments of the meal monitor app described herein.

In this embodiment, EBA sends a request through a resident API to app 804 for glucose data recently collected from the user. App 804 processes the request and supplies the queried data back to the EBA as shown in the loop depicted in FIG. 8. The EBA can associate in time the glucose data with the description of a recently consumed meal and optionally upload that meal and glucose data to trusted computer system 180 through network 190, represented herein as a central cloud based database and related services. The glucose and meal data can be categorized, indexed, and stored long term either at smart phone 120 or at the central cloud system 180, 190.

The user can access this data, for example, using an internet browser operating on a smartphone 120 or a separate computing device such as personal computer system 170 as shown here. The central cloud system 180, 190 can provide a data analytics tool via the user's web interface 806, which the user can use to enter user-specific information, adjust settings of the EBA, analyze glucose responses to meals consumed, and make informed decisions as to insulin dose adjustments or corrections. Furthermore, this data can be accessed directly by the user's HCP, either alone or in a collaborative fashion with the user during a visit, to investigate the efficacy of the user's insulin treatment and make adjustments thereto. Overall the analytics tool 806 can assist the user in long term diabetes management and integration with other therapy decisions or user engagement systems.

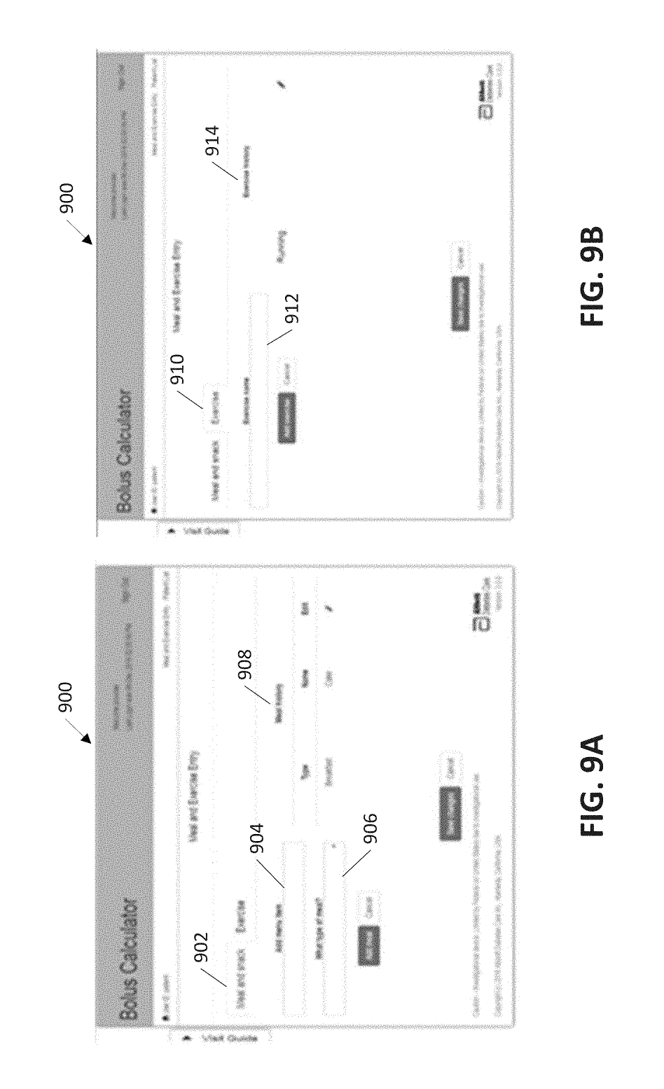

Referring now to the user-specific configuration of the EBA, FIGS. 9A-B are example embodiments of graphical user interface screens 900 displayable to a user for the entry of initial information regarding meals and exercise. The initial setup of the EBA can occur either using the smart phone app or the web-based analytics tool 806. In many embodiments, it is more convenient to perform the initial setup using the analytics tool 806, since it is easier to type into a computer keyboard than a smartphone, and FIGS. 9A-B represent user interfaces displayed via analytics tool 806, although similar information can be entered via the smartphone interface.

Here, screen 900 includes a first selectable field or tab 902 that provides a user interface that allows the user to enter a list of menu items that the user typically consumes. (Data entry can be performed by the user-patient or the HCP.) This is shown in FIG. 9A. Screen 902 can include a first data entry field 904, which in this embodiment is a free text entry field, where the user can enter a description or name of a meal that the user has or will consume. A second data entry field 906, which in this embodiment is a selectable drop down menu, can be used to select a category to which the meal belongs such as, breakfast, lunch, dinner or snack to name a few. Other standard user interface data entry techniques can be used as well.

Selectable fields are regions of the display that can be selected by the user, such as with touch in the case of a touchscreen, selection with a mouse cursor and click or touch pad tap, selection with a voice control, and other manners of selection. The selectable field as displayed on the screen can be a button, a tab, selectable text (e.g., a hyperlink), a free text entry field, a geometric shape such as an arrow indicating a drop down menu, a check box or circle, and the like. Selection of the field can result in the display of an entirely new screen, the display of a pop-up window over the current screen, the display of a drop-down menu, can allow the entry of text into a free text field, can result in selection of a displayed option, and the like. For ease of reference these fields will be referred to herein on certain occasions as tabs or buttons.

Although not shown, when entering the description of a meal menu item, the user can also enter a corresponding bolus dose that would be administered before or after consumption of the meal menu item, e.g., the meal bolus amount. This can be the amount that the patient typically takes upon consumption of the meal. Alternatively, the carbohydrates can be estimated for that meal and, based on the patient's carb ratio, a bolus calculator can be used to determine the bolus amount for that meal. However, any mechanism may be used to determine the bolus amount to enter for each meal. There are many tools available that can be integrated with, or used in conjunction with, the EBA that can help determine these meal bolus amounts, such as a bolus calculator, a food library that provides nutritional information, etc. In some embodiments, one or more of those tools are linked or otherwise made accessible here to allow the patient to make that calculation. Conveniently, this activity can be done all at once and with the assistance of an HCP or nutritionist.

Once the desired information has been entered the user can save that information, at which point it is displayed within meal menu section 908. Meal menu section 908 can list all of the entered meals for that user with a link that allows the user to edit the particular entry.

The entry of exercise information can occur by selection of tab 910 as shown in FIG. 9B. Here, a data entry field 912 allows the user to enter a description or name of an exercise activity that the user has or will perform, e.g., running, walking, swimming, biking, etc. Again, although not shown, a data entry field can be included through which the user can enter an associated bolus correction to compensate for that particular exercise. This can be an amount that the user typically subtracts from a prior rapid acting insulin does to compensate for that activity. Alternately or in addition to this, a data entry field can be included through which the user can enter an amount of carbs to inject or consume to compensate for that particular exercise. When the desired information is entered, the user can save the information at which point it will be displayed within the exercise history section 914. The exercise history section 914 can list all of the entered exercises for that user with a link that allows the user to edit the particular entry.

In some embodiments an additional setup menu is provided that allows the user to enter additional system parameters either through the smart phone user interface or the analytics tool 806. These additional system parameters can include the patient's insulin sensitivity, insulin action time, titration amount in insulin units, titration algorithm parameters (LLG sensitivity, eA1c goal), and the like.

Depending on which interface the setup is performed on, the entered parameters (meal menu, exercise history, etc.) can then be synchronized with the other components of system 100. For example, if the parameters are initially entered via the analytics tool 806, then those parameters can be pushed to smart phone 120 using conventional techniques.

Alternatively, if the parameters are initially entered via the smartphone interface than those parameters can be uploaded to central cloud system 180, 190.

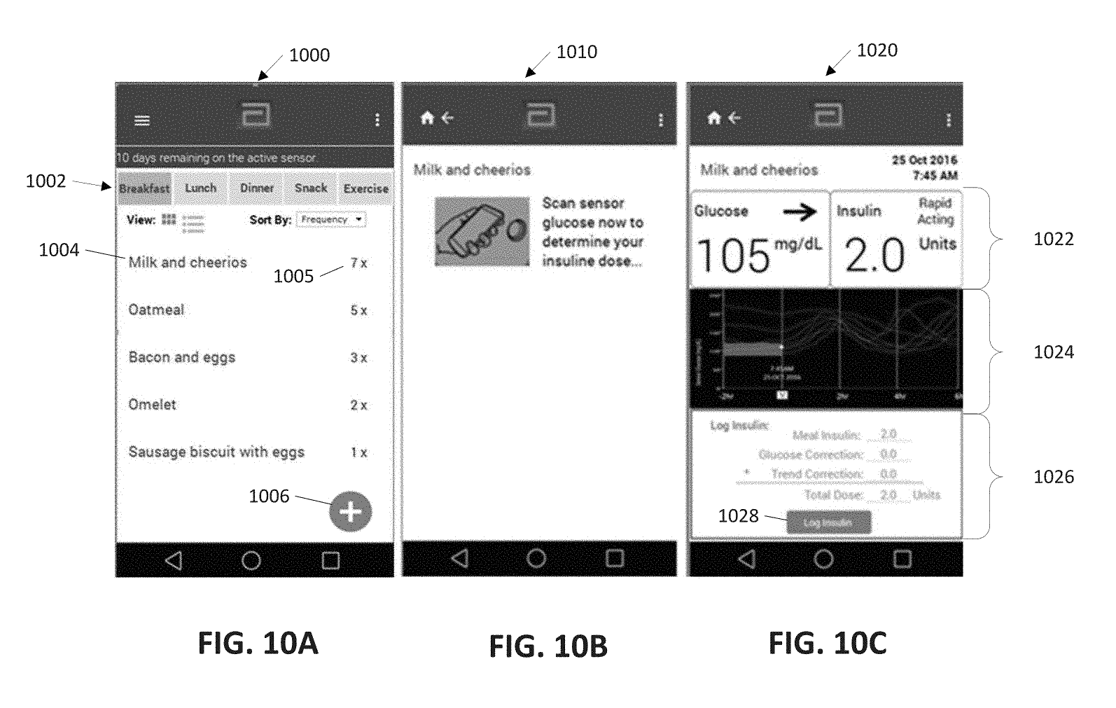

Turning now to operation of the EBA on smart phone reader 120, FIGS. 10A-C depict example embodiments of user interface screens 1010, 1020, and 1030 displayable to the user via the smartphone interface. FIG. 10A depicts an example embodiment of a home screen 1000. Near the top of home screen 1000 are a series of selectable tabs 1002 corresponding to the various categories or types of meals and exercise. The active tab can be determined by the app based on the current time-of-day. For example, if the current time-of-day is between 2 am and 10 am, then the breakfast tab is active. If the patient desires a different tab then that tab can be selected, e.g., for a different meal that the patient is about to consume.

Selection of one of the tabs causes the display of the associated items corresponding to that tab, where those associated items are also selectable. Here, the tab corresponding to breakfast has been selected and a list of menu items 1004 is displayed. Each menu item 1004 can be displayed along with a corresponding frequency or number of instances 1005 that the menu item 1004 has been selected in the past by the user. The menu items 1004 can be sortable by frequency, most recent to least recently selected, alphabetically, or otherwise.

Then the user can then select the meal that the user is about to consume to initiate the process of determining the appropriate bolus, which in this example is “Milk and cheerios.” Selection of a meal can initiate the process of collecting or retrieving the user's recent glucose data. For example, if system 100 is configured as a flash or on demand system, then a notification can be displayed to the user via screen 1010 that prompts the user to scan sensor control device 102 to retrieve the current glucose data. The collection of that data can be managed through the sensor interface app 804 and provided to the EBA as described with respect to FIG. 8. Alternatively, the EBA can include the scan routines, interface with the reader's scan transmitter hardware and software, and perform the scan itself with or without the assistance of app 804.

If reader 120 is already in possession of the user's current glucose data, e.g., because the sensor control device 102 was recently scanned or because the user's glucose data is automatically and continually uploaded to reader device 120 from sensor control device 102, then the display of screen 1010 can be omitted and selection of the meal in screen 1000 causes immediate transition to the display of an insulin logging screen 1020. Instead of the calculator using sensor glucose data, the calculator can be configured to use SMBG (self-monitoring of blood glucose) data provided by user entry or electronically, as is commonly available. Here the logging screen 1020 is presented without the prompt for the sensor scan.

An example embodiment of insulin logging screen 1020 is depicted in FIG. 10C. Here, a date and time stamp 1021 of the meal description (“Milk and cheerios”) is included at top. Below that is a first section 1022 of screen 1020 where the user's current glucose level and current trend arrow is shown followed by the recommended meal bolus insulin amount, which in this example is 2.0 units of rapid-acting insulin. This bolus insulin amount can be either the meal bolus amount that was originally entered with this meal during initial setup (or during the first time this meal is logged) or the last confirmed meal bolus amount injected or infused for this meal.

A second section 1024 of screen 1020 can display the user's current glucose data in the form of a trace on a glucose vs. time graph. The current trace can be highlighted with respect to other traces also displayed on the graph, where those other traces map glucose data collected around prior instances of the time of consumption of this same meal (e.g., milk and cheerios). The graph includes both pre-prandial and post-prandial data and each trace can span a multi hour period, which, in this embodiment begins at two hours prior to start of consumption of the meal and ends at six hours after start of consumption of the meal. This overlay provides the user with a readily digestible indication of how the user's glucose has responded to the consumption of this meal and the administration of insulin on prior occasions.

These traces can be aligned by the associated meal time stamp, where the time-stamps are aligned with zero on the x-axis (time axis). These traces can be displayed with the actual glucose values plotted against the Y-axis (glucose axis) as shown here, or in other embodiments the traces can be normalized (vertically offset) such that each trace intersects the current glucose value for the user (e.g., 105 mg/dL) at the time where prior instances of this meal were logged. In still other embodiments, the traces can be shifted in time such that each trace has the same Y-value (e.g., 110 mg/dL) at time 0 on the x-axis.

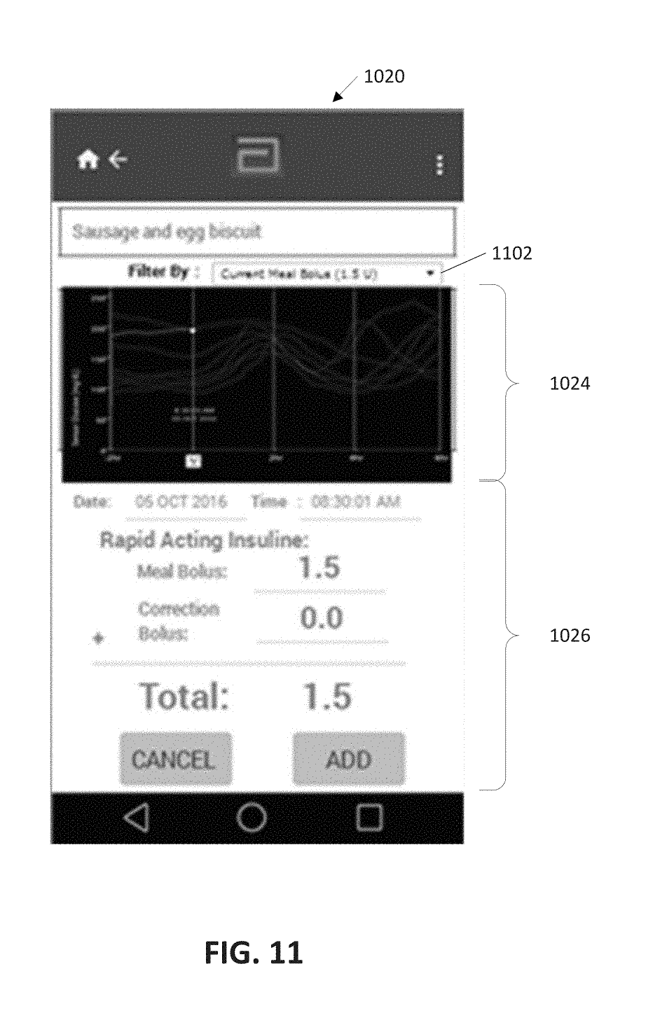

The other historical glucose traces can be included by default based on those instances of the same prior meal having actual meal bolus doses that are nearest to the current recommended dose. Additional filters can be provided to further select which traces to include in the graph such as, for example: a) alternate instance where the same actual dose was recommended; b) instances where one or more similar exercise activities were also logged within 12 hours of the meal; and/or c) in cases where the user has selected an exercise activity through screen 1000, then instances where one or more similar meals were also logged within a predetermined time period (e.g., 6 hours, 12 hours, 24 hours) of the exercise activity. FIG. 11 depicts another example embodiment of insulin logging screen 1020 where section 1022 is omitted. A drop down filter field 1102 is included to allow the user to identify the characteristic or parameter by which is the EBA selects the other historical traces to display alongside the user's current glucose trace in section 1024.

Referring back to FIG. 10C, a third section 1026 of screen 1020 breaks down the recommended bolus dose by component. In some embodiments, each value in section 1026 can be modified by the user. In this embodiment, the first component is the “meal insulin” amount, which can be the most recent bolus amount confirmed for this particular meal, or if there wasn't a recent value confirmed, then the meal insulin amount entered during initial setup can be used.

Various correction calculations are available and can be employed by the EBA. These corrections can take into account the patient's insulin sensitivity setting, but can also take into account glucose level, glucose trend, insulin action time, and insulin-on-board. The correction fields may be editable so the patient can determine their correction amount using a means separate from the EBA or make adjustments to those corrections suggested by the EBA. As shown in FIG. 10C, beneath the “Meal Insulin” amount are editable fields for the “Glucose Correction” amount and the “Trend Correction” amount, which correspond to the insulin correction amounts that compensate for the user's current glucose level and current glucose trend, respectively. Other correction components can be included as well such as one for insulin on-board. In this embodiment, these correction fields are initially calculated by the EBA. Alternatively, these correction fields in section 1026 may be combined into one correction field.

The sum total of these components results in the “Total Dose” recommended by the EBA. If the user edits any one of these three component fields then the total dose amounts will automatically update to reflect the user edit. In some embodiments, the original EBA recommendation will remain displayed in section 1022, while in other embodiments the dose recommendation in section 1022 will update to reflect any user changes within section 1026.

An additional user selectable field 1028 is provided (e.g., “Log Insulin”) that allows the user to confirm the insulin dose actually administered as reflected in the “Total Dose” field, along with the various components. Selection of field 1028 logs the information represented in section 1026 and associates the log information with this meal, the time and date of consumption, and the glucose data from this peri-meal time period (e.g., from two hours before to 6 hours after).

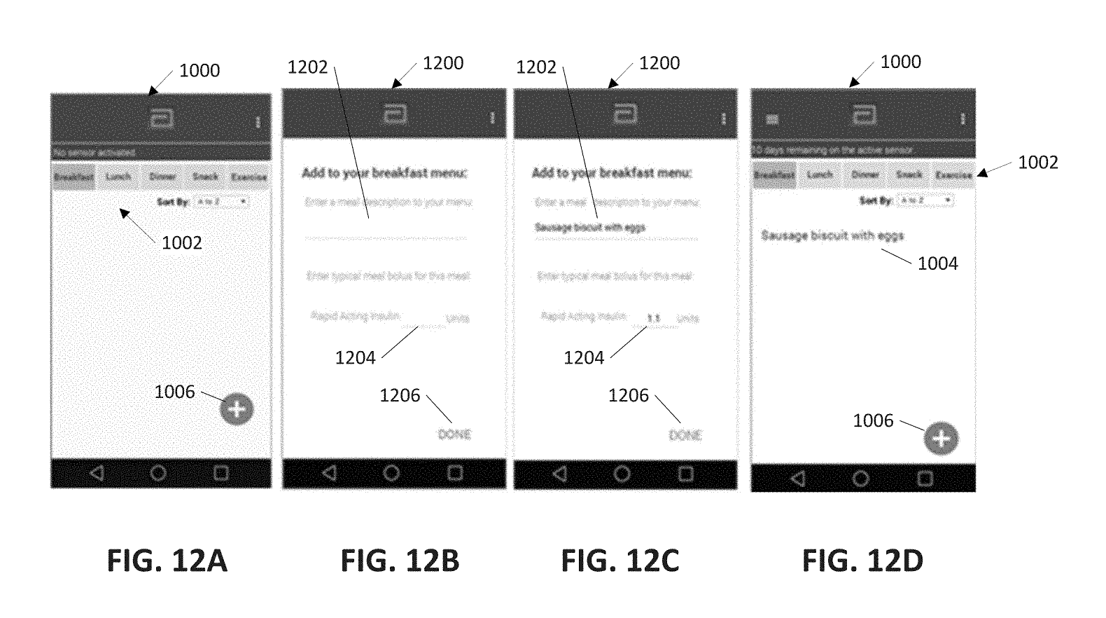

As mentioned with respect to FIG. 10A, home screen 1000 can include a selectable field 1006 that allows the user to add a new meal or activity. FIG. 12A depicts an example embodiment of home screen 1000 where the breakfast category of meals has not yet been populated with a menu item. Selection of field 1006 causes the display of a menu item entry screen 1200 as depicted in FIG. 12B. Screen 1200 can include a first data entry field 1202 in which the name or description of the meal to be added can be provided. Screen 1200 can also include a second data entry field 1204 where the meal bolus amount can be entered. (In other embodiments, the type of medication or insulin can also be specified.) As described earlier, the determination this meal bolus amount can occur through collaboration of the user with his or her HCP or through the use of a bolus calculator.

FIG. 12C depicts screen 1200 after entry of the meal description (“Sausage biscuit with eggs”) and the meal bolus amount (1.1 units). Upon entry of the desired information the user can complete the data entry process by selecting field 1206 (“Done”). At this point, home screen 1000 is displayed again with the recently added menu item 1004. The user can then select that menu item and proceed to log an insulin dose to establish a history for that meal.

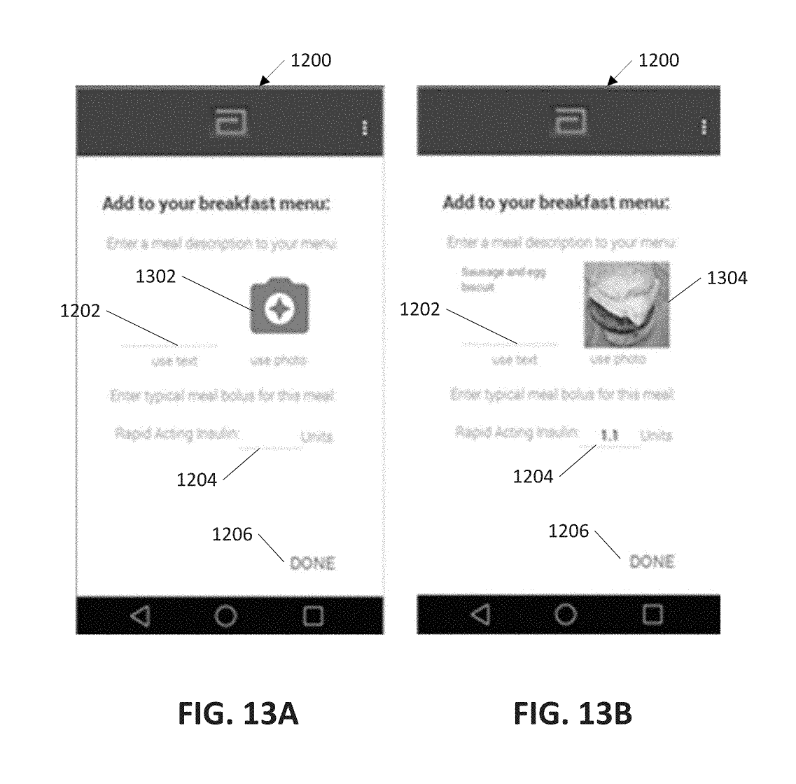

The EBA can also be configured to allow the user to describe meals and activities with the use of photos or other images. FIGS. 13A-B depict example embodiments of meal entry screen 1200 that are similar to those described with respect to FIGS. 12B-C, but that also have the capability to enter a visual description of the meal. In FIG. 13A, screen 1200 is shown with an additional user selectable field 1302. Selection of this field 1302 prompts the user to identify a photo or image, either by selecting one from the smart phone's photo library or by taking a photo of the meal using the smart phone's camera. FIG. 13B depicts screen 1200 after the user has entered the identification of the meal (“Sausage and egg biscuit”) and bolus amount (1.1 units) and identified a photo 1304 depicting the meal. The use of photos has the potential to be easier to use, and may appeal to those users for whom a one time setup of the meal menu along with consultation with an HCP is not possible.

Although not shown, in some embodiments meal entry screen 1200 can include a selectable field that allows the user to enter a second similar meal, where that similar meal may be share one or more ingredients with the meal just entered (e.g., sausage biscuit with eggs is similar to a sausage and egg biscuit). When this field is selected, the meal entry screen is prepopulated with the same meal description and bolus amount to allow for easy editing by the user. Likewise, in some embodiments, home screen 1000 can also include a field that allows the user to enter a similar meal. Selection of that field will cause only the meal tabs 1002 to be displayed (excluding the exercise tab), at which point the user can select the tab and corresponding meal from the menu that is similar to the new meal to be added. When the meal item is selected then the meal entry screen 1200 is displayed and prepopulated with the meal bolus amount that is the most recent actual meal bolus amount associated with the selected similar meal. From this point the user can then enter a meal description and/or image and then add the meal to the menu.

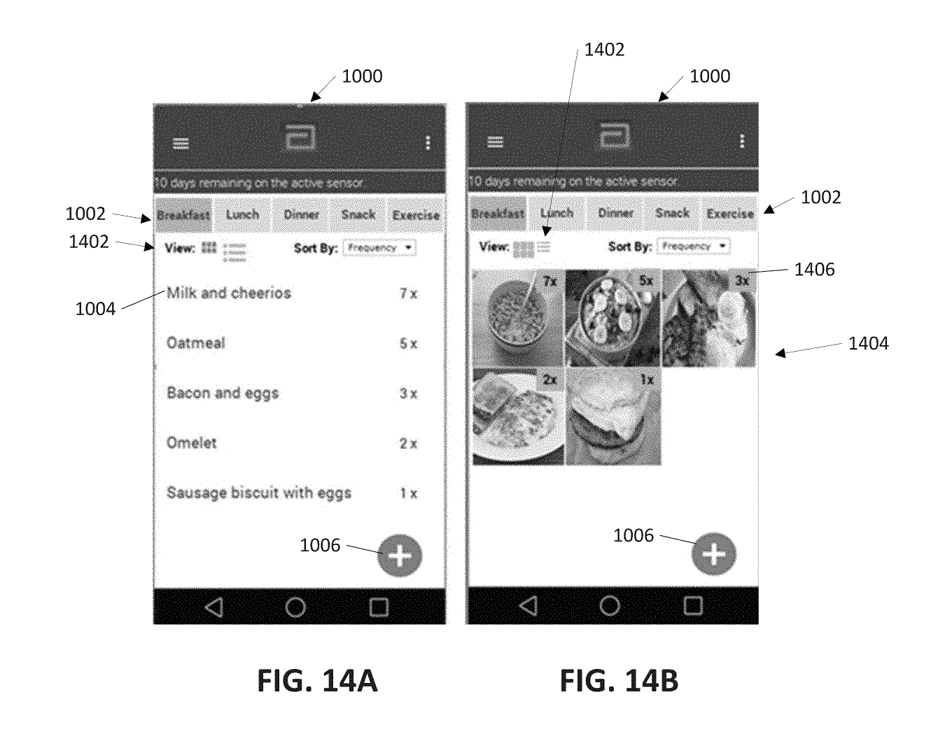

When one or more meal images have been captured and associated with a meal, the user can choose to view menu items by image as opposed to (or in addition to) a textual description of the meal. FIG. 14A depicts an example embodiment of home screen 1000 with a selectable field 1402 that allows the user to toggle between the text-only menu display of FIG. 14A and an image-only menu display of FIG. 14B. In FIG. 14B, each menu item is shown using only that menu item's image as a series of tiles 1404 arranged in rows and columns. The addition of new meal images further populates the display and the user can have the option of scrolling up or down as necessary. In this embodiment, each image tile 1404 is displayed with a textual indicator 1406 of the number of previous instances where that meal has been logged.

After every new meal is logged, the actual meal bolus amount and the associated glucose trace data are recorded. The EBA can then process the post-prandial glucose data associated with this particular meal to determine if a titration recommendation is warranted. In one example embodiment of determining a titration recommendation, the process can retrieve every glucose data set over a predetermined post-prandial time period (for example, from one half-hour post meal time stamp to six hours later or the next meal time-stamp, whichever is sooner) for the same meal as to which the most recent meal bolus amount was administered. These individual glucose data sets (e.g., traces) may be further filtered by only including those associated with the prior activity state of the most recent meal. The process can have a minimum threshold of glucose data sets to proceed—three for example. Each trace can be offset such that the starting value of the trace is a predetermined glucose value (e.g., 110 mg/dL) and the data from the traces can be combined to create a post-prandial glucose data set group for that menu item.

The post-prandial data set group for that menu item can then be processed using hypoglycemia risk and/or variability determination techniques described in U.S. Patent Publication No. 2014/0350369, which is incorporated by reference herein in its entirety for all purposes. For example, if the process determines that hypoglycemia risk is relatively high (e.g., above a threshold for high risk), then the EBA can generate a recommendation to reduce the meal bolus amount. If the process otherwise determines that the hypoglycemia risk is low (e.g., below a high threshold or a moderate threshold) and a glucose central tendency (e.g., the glucose median or mean) is above a central tendency threshold (or some other indication of the need to generally lower glucose values), then the EBA can generate a recommendation to increase the meal bolus amount. If the process otherwise determines that the hypoglycemia risk is moderate (e.g., above a threshold for moderate risk or above a threshold for low risk and below a threshold for high risk), but the glucose central tendency is above the central tendency threshold, then the EBA can generate a recommendation to reduce glucose variability. If the process determines that the data are insufficient to reliably determine hypoglycemia risk, then the software can do nothing or take no action in response. In some embodiments, the recommendation to increase or decrease will be a specific meal bolus or increment amount as preconfigured in the EBA; for instance, if preconfigured to titrate by 2 units then the recommendation could be to reduce the meal bolus by 2 units or, if the original amount is 15 units, then the recommendation could be to change the meal bolus to 13 units.

In one example embodiment, the hypoglycemia risk determination is to calculate a central tendency value (e.g., median) and a variability value (e.g., the median—10th percentile) for the post-prandial glucose data set group. A high hypoglycemia-risk threshold can be retrieved from memory (e.g., from a lookup table of thresholds vs variability, where the calculated variability value is input, and the threshold is output). The central tendency value can then be compared to this threshold: if the central tendency is below the threshold, then a recommendation to reduce the meal bolus can be generated and output and displayed to the user.

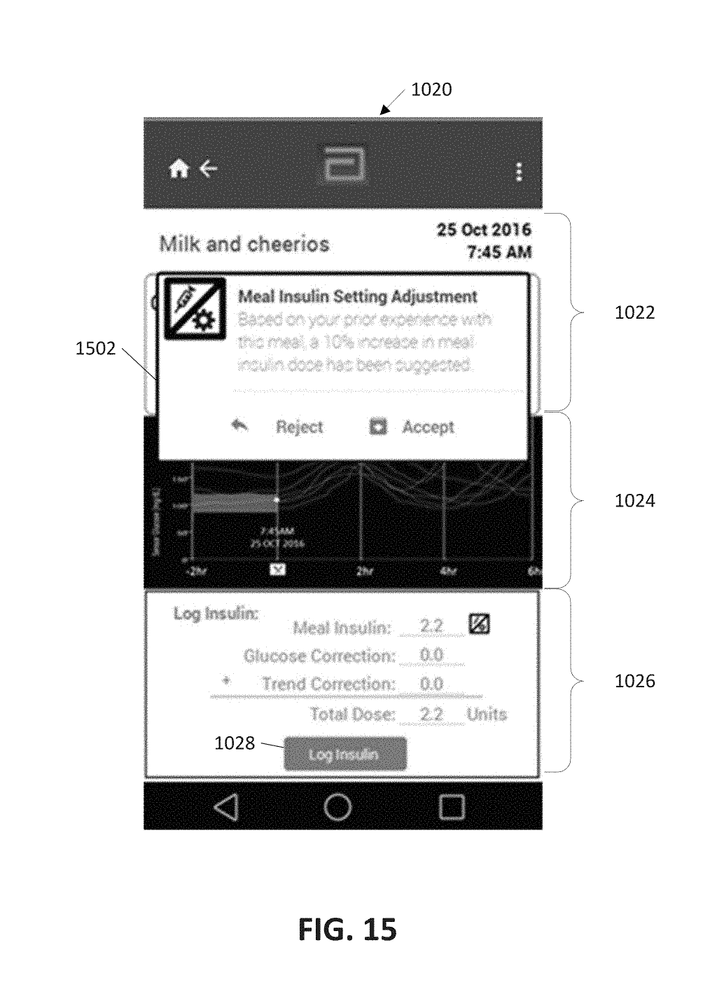

Otherwise, a moderate hypoglycemia risk threshold can be retrieved from memory (e.g., a lookup table of thresholds vs variability, where the calculated variability value is input, and the threshold is output). The central tendency can then be compared to this threshold: if the central tendency value is above this threshold and above a predetermined threshold defined by a retrieved eA1c goal setting (e.g., 154 mg/dL for 7% eA1c), then a recommendation to increase the meal bolus can be generated and output and displayed to the user. An example of this recommendation output is shown as notification 1502 on insulin logging screen 1020 in FIG. 15. The user has the option of accepting or rejecting the recommendation. If accepted, the meal bolus amount recommendation will be used as the meal bolus insulin amount and repeated the next time the meal is selected. If rejected, then no modification will be made and the EBA will revert to the last used meal insulin amount (i.e., the meal insulin will change from 2.2 to 2.0 in FIG. 15).

If the central tendency is below a moderate hypoglycemia risk threshold and above a predetermined threshold defined by the retrieved eA1c goal setting (e.g., 154 mg/dL for 7% eA1c), then a recommendation to address variability can be generated and output and displayed to the user.

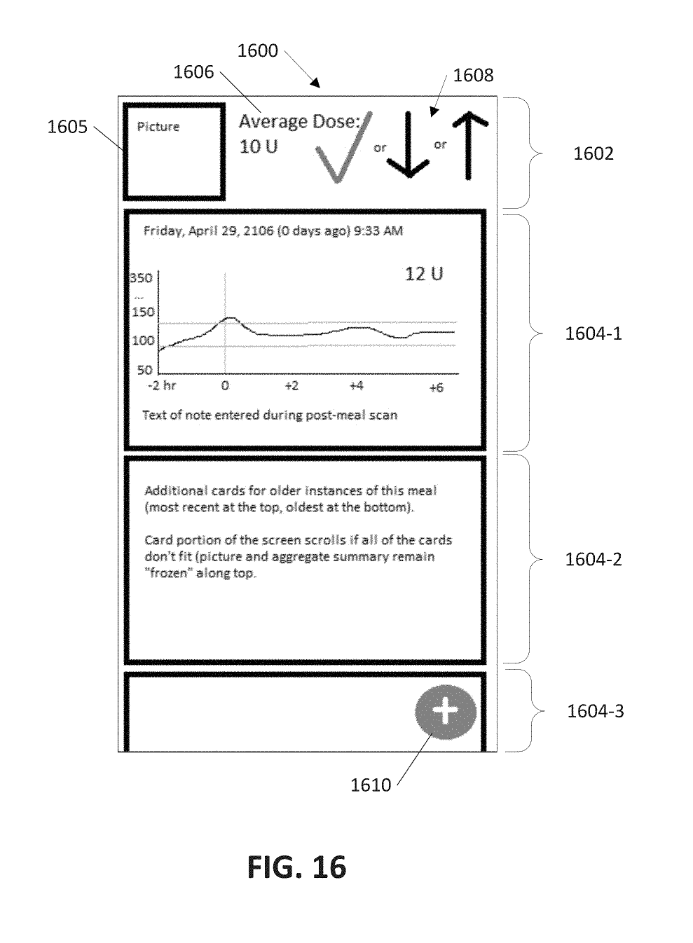

As with the meal monitor application, the EBA can include the functionality to generate one or more reports for the user to assist in interpreting and understanding the collected data. FIG. 16 depicts an example embodiment of a meal history report 1600. Report 1600 provides an easy way to reference a summary of the collected data for a particular menu item. A first section 1602 of report 1600 can include a picture 1605 of the meal as a thumbnail (clicking on the thumbnail can cause a full size photo to be displayed), alongside an average dose 1606 for this meal (which in some embodiments can be weighted to count more recent doses more heavily than older doses), and an icon 1608 indicating the average post meal response in the past, where a checkmark indicates the post-prandial data is generally in a target range, a downward arrow indicates post-prandial data is generally beneath a target range, and an upward arrow indicates post-prandial data is generally above the target range. A variety of known methods for evaluating hypoglycemia risk can be used for this purpose.

Below section 1602 can be another series of sections or cards 1604, where each card 1604 corresponds to a meal instance of this type. Each card 1604 can have a time stamp, a bolus that was administered, and a graph showing the data for a peri-meal time period (e.g., from a pre-prandial time to a post-prandial time). Each card 1604 can show the patient their glycemic response for the given meal for specific insulin dosages to provide the patient with information and feedback on how much to decrease or increase the bolus dose.

The EBA can monitor to determine if all post-prandial glucose data has been collected and, if some or all data has not been collected, then prompt the user to obtain that data, for example by scanning sensor control device 102. In some embodiments, the EBA can have the ability to start and scan sensor control devices 102. The EBA will automatically associate glucose data within the peri-meal time period with the instance of the meal. If the peri-meal data contains a glucose excursion or episode, then a notification can be generated prompting the user to enter a free text note to provide added context for that instance of the meal, or that note can then be displayed within the respective card 1604. This note two can also be entered when the user creates the meal instance or at the user's own initiative later in time (i.e., without prompting by the EBA).

Screen 1600 can include a selectable field 1610 for entering new instances of a meal of this type. Selection of this field will cause the insulin logging screen 1200 to be displayed (see, e.g., FIG. 10C), from which the user can review current glucose data and determine the appropriate bolus dose.

Many of these embodiments can prove beneficial for Insulin Using Patients (IUPs) that use variable dosing for meals. The dosing response and/or insulin sensitivity varies with other factors such as exercise. Further fine-tuning can be facilitated by using the card notes entry as a tag where the EBA has the ability to filter on a specific tag within the same menu item. In these embodiments, on the meal history screen 1600 the summary header section 1602 can then be updated by only considering the instances of this meal that fit the criteria of the filter. For example, the user might tag each meal instance of a particular type with “post run” when the user ran or jogged prior to the meal. This exercise prior to the meal might impact the insulin sensitivity subsequent to the run, and by having the ability to filter on this tag, the user will receive additional fine-tuning guidance for insulin dosing with this meal and exercise combination.

In some embodiments, the user interface of reader 120 can be streamlined for meal-time dosing for a subset of IUPs that rely on fixed dosing for meals. The goal for these users can be slightly different in that they are more routine driven and may care less about what they eat and more about adjusting their meals to fit certain insulin doses. For these fixed dosing IUP users, home screen 1000 could include simply three user selectable fields (e.g., tabs, icons, or photos) of breakfast, lunch, and dinner. Meal history screen 1600 could be configured to show the best or optimal meals for a certain dosage, or to show a ranking of different meals with respect to the dosage based on the degree in which those meals fall within the target glucose range for the user. General classifications for ranking meals can include best or optimal meals for a certain dosage, not enough food for a certain dosage, and too much food for a certain dosage.

In some embodiments, the EBA can be configured to accommodate different IUP users. For example, the EBA can provide the user with the option of selecting or instituting a setting that determines whether the EBA will use an algorithm that allows for fine tuning of insulin dosing for the meal (for variable dosing IUP's), or ranking the best or optimal meal alternatives for a given insulin dose (for fixed dosing IUP's). In other embodiments, different versions of the EBA could be supplied for different user populations such that the algorithm was could not be toggled.

In other embodiments, the EBA can be configured for use by non-diabetic (ND) and/or non-insulin using patients (NIUP). In these embodiments, the EBA can provide meal glucose response data in terms of “Glucose Loading” and meals could be ranked by their respective glucose loading and displayed accordingly in the meal history screen 1600. In these embodiments the home screen 1000 can be configured to show the cumulative daily total glucose loading to advise the user how much glucose loading allowance is left based on a preset restriction or baseline, for any given moment of a given day. ND and NIUP users could use these embodiments of the EBA as a diet management tool. When enough meals have been categorized, the EBA can provide meal recommendations for different “Glucose Loading” allowances based on different types of restrictions that the users may want to set (e.g., a per day restriction, a per meal restriction, or a per meal of the day (breakfast, lunch, or dinner) restriction).

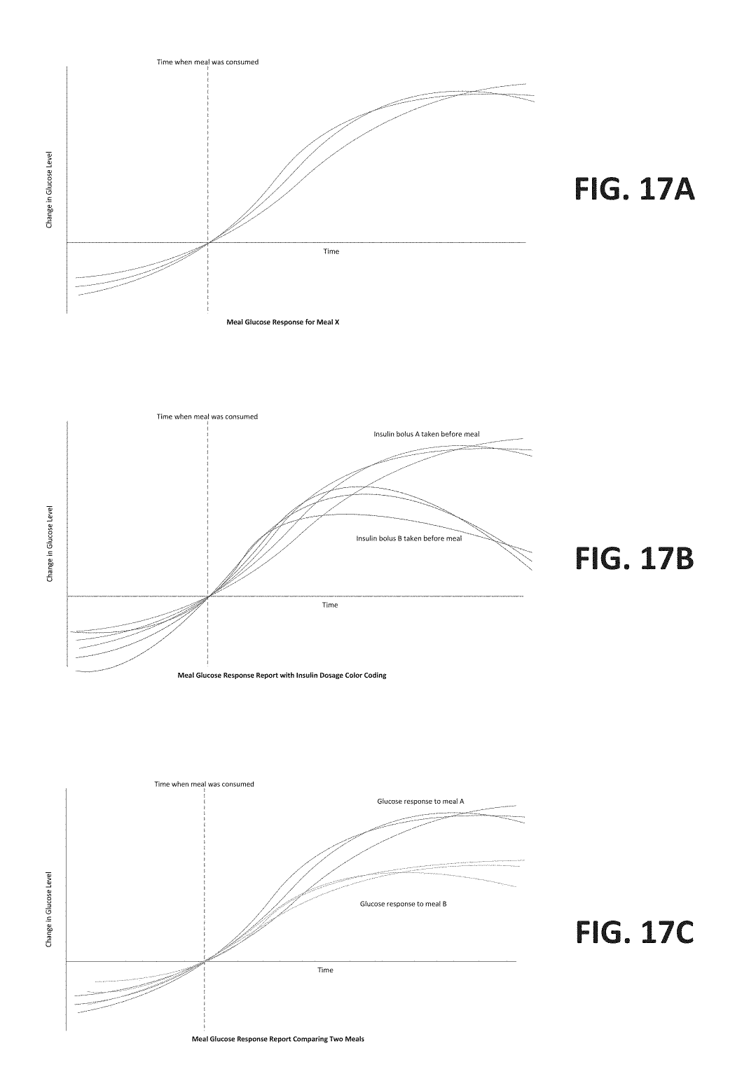

The EBA can be configured to generate another report that is displayable to the user, referred to herein as the Glucose Response Report (GRR). Example embodiments of the GRR are depicted in FIGS. 17A-C. The GRR can allow a patient's glucose response to be empirically estimated by overlaying the glucose response to a given meal (or exercise activity) over the numerous times this meal was consumed (or exercise activity performed) as shown in FIG. 17A. All traces can be normalized to the time of the meal to assess the relative glucose pattern thereafter. For a given patient, the glucose response to a given meal is expected to be similar each time it is consumed. In alternative embodiments, the traces can be displayed as an Ambulatory Glucose Profile (AGP) that is again normalized such that the relative origin of each trace is set at the time of the meal. AGP graphs are described in detail in U.S. Publ. No. 2014/0350369, which is incorporated by reference herein in its entirety for all purposes.

The effect of different insulin bolus dosages to a given meal may be compared by using the GRR and coding (e.g., by color or pattern) the glucose traces for the insulin dosages as shown in FIG. 17B. The coding enables the HCP to visually compare the effect of each insulin dose on the glucose response and adjust the patient's insulin dosage by interpolating or extrapolating from the traces in the graph.

The effect of different meals on a patient's glucose response may be compared by using the GRR and coding the glucose traces (e.g., by color or pattern) for each menu item as shown in FIG. 17C. The coding enables the HCP or patient to visually compare the patient's glucose response, and provide input to meal selection and menu planning to optimize the patient's glycemic control. The average glycemic response to a given meal may be statistically calculated over the number of times the meal was consumed.

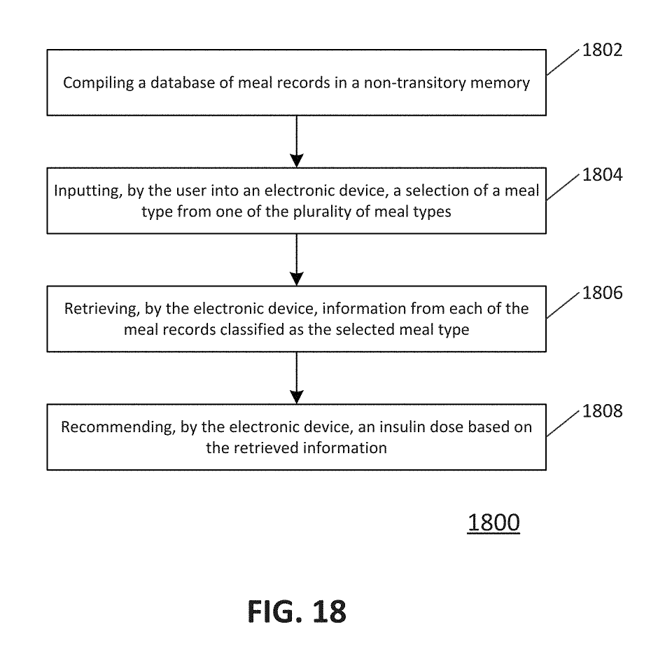

FIG. 18 is a flow diagram depicting an example embodiment of a method 1800 of using the EBA from the perspective of implementation by system 100 and the user, and is useful in illustrating the many variations of system 100 and method 1800. In this example, method 1800 is used to determine an insulin dose for administration with the consumption of a meal. At 1802, a database of meal records is compiled in a non-transitory memory. The meal records can be compiled through the original entry of a meal record into the EBA, e.g., using either the web-based tool 806 or the mobile app 802. The non-transitory memory can be located on one device or distributed over one or more devices, for both cases examples of the device include, but are not limited to, reader 120, drug delivery device 160, an integrated reader and drug delivery device, personal computer system 170, trusted computer system 180, network 190 and/or other combinations thereof. If distributed over multiple devices, the devices can be configured to upload the meal records (or portion thereof) to a database at a central location, such as trusted computer system 180, from which the databases of all devices can be matched.

Each meal record in the database corresponds to a meal previously consumed by the user (e.g., diabetic patient) and includes information representative of an insulin dose administered with that previously consumed meal. For example, referring back to FIG. 10A, the meal record can correspond to a single instance where any one of the menu items 1004 was consumed (e.g., an instance where milk and cheerios was consumed, an instance where oatmeal was consumed, etc.). Each meal record in the database is classified as one of a plurality of menu items 1004 (e.g., Milk and cheerios, Oatmeal, etc.), which can serve as an identifier for that class of meals under which all instances of consumption of that meal can be classified (e.g., all meal records for instances where oatmeal was consumed can be classified under the menu item of “Oatmeal”).

Each meal record can include a description of the meal, the context in which the meal was consumed, an image of the meal (see, e.g., FIG. 13B), and/or any other note or information relevant to that moment in time and the user's diabetes management. Each meal record can also include a date and/or time of consumption of the meal (e.g., a time stamp of the start of the meal; see, e.g., date and time stamp 1021 of FIG. 10C) and glucose data of the user from a time period occurring before and/or after (e.g., corresponding to) the time of consumption of the meal (see, e.g., glucose data depicted in sections 1022 and 1024 of FIG. 10C). In some embodiments, the meal record can be further classified by meal type based on time periods of the day (e.g., breakfast, lunch, dinner, etc.).

Next, at 1804, method 1800 includes inputting, by the user into an electronic device (e.g., reader 120, drug delivery device 160, an integrated reader and drug delivery device, personal computer system 170, and the like) a selection of a menu item (e.g., Milk and cheerios) from one of the multiple menu items (e.g., Milk and cheerios, Oatmeal, Bacon and eggs, etc.). This selection can be input into screen 1000 (see FIG. 10A) or other meal selection screen displaying a list of menu items 1004 under a particular meal type heading 1002. The meal selection screen can also display the indication of the frequency that each of the menu items 1004 has previously been consumed by the user. Then, at 1806, method 1800 includes retrieving, by the electronic device, information from each of the meal records classified as the selected menu item 1004. This retrieval can be from a database stored locally on the device, or can be include downloading the data over a communication link, e.g., from trusted computer system 180 and network 190 to reader device 120 over link 142 (see, e.g., FIG. 1). The entirety of the records need not be retrieved and in some embodiments only that portion of the record that is relevant for that next action in that particular embodiment is retrieved.

At 1808, the next action of method 1800 includes recommending, by the electronic device, an insulin dose based on the retrieved information. In some embodiments, this action includes determining, by the electronic device, the recommended insulin dose and displaying the recommended insulin dose to the user on a display of the electronic device. In some embodiments, the retrieved information includes only the most recently administered insulin dose for the selected menu item and the recommendation is based only on that most recently administered insulin dose and one or more correction parameters if available. In some embodiments, the recommended insulin dose in made based on at least two dose components, and the recommended insulin dose is displayed to the user with at least two dose components. For example, the embodiment described with respect to FIG. 10C displays three dose components and the embodiment described with respect to FIG. 11 displays two dose components. The method can include receiving a modification, at the electronic device and entered by the user, to at least one of the at least two dose components, in which case the method can also include updating the recommended insulin dose based on the received modification to the at least one of the at least two dose components. All embodiments of method 1800 can include actually administering the recommended insulin (or other medication) dose, or other dose (whether recommended or not) based on the recommendation made. The administration of the dose (bolus or basal) can occur by injection with a syringe, infusion with a drug delivery device, or in any other desired manner.

The display of the recommendation can be accompanied with the user's glucose data, such as shown in FIG. 10C. The user's glucose data can be displayed on a graph with additional glucose data corresponding to one or more previous instances of consumption of the meal of the selected menu item.

As described herein, the EBA can be particularly beneficial in allowing the user to determine a meal correction bolus dose without estimating or calculating nutritional content of the meal, such as the meal's carbohydrate content. Thus, in many embodiments (but not all), one, more than one, or every meal record can be stored in the database without quantitative information indicating nutritional content of the meal, such as carbohydrate content, fat content, sugar content, calories, protein content, any combination thereof and the like. In many embodiments, such content values are unnecessary because the EBA relies on the repeatable nature of the user's meal consumption habits, where variations in nutritional content between instances of consumption of the same menu item are minimal, negligible, or otherwise non-existent. In other words, when a user regularly consumes milk and cheerios on a daily or weekly basis, the amount of milk and cheerios consumed each time is generally constant as users tend to develop consistent habits and regular dietary patterns. It follows then that the portion of the bolus dose based on nutritional content alone can remain constant. This portion of the bolus dose can be set initially based on the recommendation of the HCP or through the use of a bolus calculator that relies on nutritional content or in another fashion.

After using the EBA repeatedly over a period of time, adjustments to that dose (other than corrective portions of the dose to compensate for insulin on-board, current glucose level, current glucose trend, etc.) can be made experientially by reference to the user's glucose responses and titrating accordingly. Thus, nutritional content, especially carbohydrate content, can be omitted or absent from the meal records and not displayed at any point to the user. In many embodiments, the recommendation of the insulin dose or a recommendation to titrate the dose is calculated or determined by the EBA without reference to the carbohydrate content or any other nutritional content of the selected menu item as the EBA is blind to the nutritional content of the consumed meal.

### Embodiments of Meal or Menu Planning

In the embodiments described herein, a database may be created for the given patient by collecting the patient's glycemic response to many meals over time. Combining the meal's glycemic response with information on the meal content (e.g., from the ingredients in the recipe used to prepare the meal or from the nutritional information available on restaurant meals) the patient's glycemic response to a new recipe or restaurant meal may be estimated by comparing the ingredients or nutritional information of the new meal with meals in the patient's database. In this manner, a weekly menu may be planned algorithmically by selecting from the meals the patient has consumed as well as adding new menu choices by extrapolating the patient's glycemic response based on similar meals for which there is data on the patient's glycemic response.

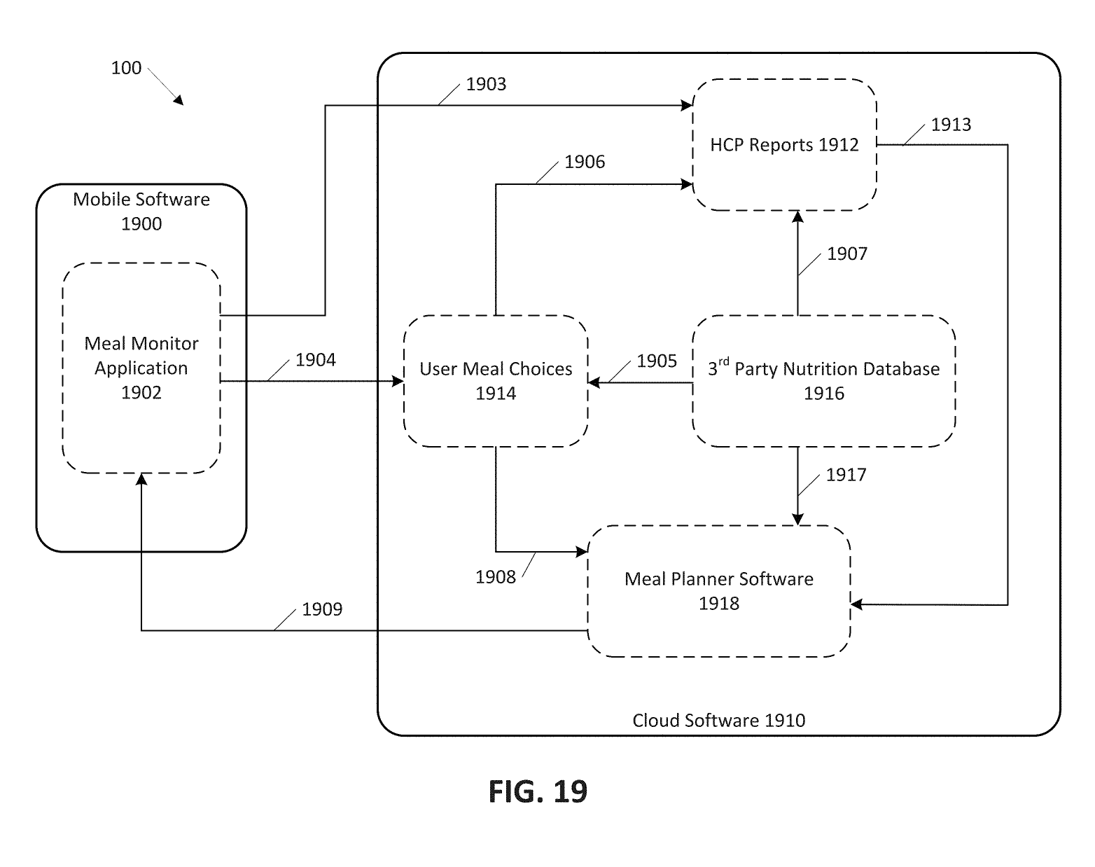

An example embodiment of such a meal planning architecture is depicted in FIG. 19, which depicts informational flows between various informational (stored information), software and hardware components of system 100. The software components are composed of multiple instructions stored on non-transitory memory that, when executed by processing circuitry, cause the processing circuitry to take various actions. Although separate components are shown, the various components can be executed on shared processing circuitry. An example embodiment of a meal monitor application 1902, which can include the EBA, is operating in conjunction with mobile software 1900, such as a smartphone operating system. This embodiment can be implemented for both IUPs and NIUPs, and the mobile software 1900 can be implemented on a reader 120 or non-reader device.

Meal monitor application 1902 can communicate information to and receive information from various software and hardware entities that, in this embodiment, form cloud-based software 1910. In some embodiments, these software entities can be accessed through network 190 of FIG. 1. Here, cloud-based software 1910 includes HCP report generating software 1912, user meal choice storage and/or processing 1914, a nutritional database 1916 (which in this embodiment is operated by a third party), and meal planner software 1918.

Meal monitor application 1902 can be used by the individual user, e.g., in those manners described herein, to compile a list of meals in text or photos, to collect the user's glucose level data, to collect the user's selection of meals consumed from the provided list, and to collect the user's insulin data, if any. The user's glucose data, meal choices, and/or insulin data can be communicated to HCP report generating software 1912 over information path 1903. Meal monitor application software 1902 can also transfer the list of meals in text or photos, including new meal information whenever entered, to user meal choice storage and/or processing software 1914 via information path 1904. The information paths or flows referenced in FIG. 19 represent only the flow of information and, while they can correspond to actual physical wired and/or wireless communication links, the presence of such physical communication links in the manner shown is not required. The arrows shown on the various paths in FIG. 19 represent a primary direction of information flow, and information can flow in the opposite direction as well (i.e., each information path can be bidirectional).

Many different nutritional databases 1916 can be present, but for simplicity one is shown. Nutrition database 1916 is, in many embodiments, operated by a third party provider of meals or recipes and can store these various recipes with nutritional data (e.g., carbohydrate content, sugar content, fat content, protein content, calories, portion sizes, and the like). Also, or alternatively, nutrition database 1916 can store nutritional data for meals commonly served by one or more restaurants. The user can selectively add this nutritional data from database 1916 to the user meal choice software 1914 via information path 1905, for example, for meals that the user has or is planning to consume.

HCP report generating software 1912 can have access to the information in the user meal choice software 1914 and nutrition database 1916 via information paths 1906 and 1907, respectively. HCP report generating software 1912 can generate a report for each meal choice, where the report can include, for example, an AGP representation of the glucose data for each meal choice. HCP report generating software 1912 can also perform and display an analysis of glucose response to each meal for the given patient, which may include heuristics generated over time and a library of meals and their glucose response. HCP report generating software 1912 can determine and output a ranking or categorization of the user-selected meals by glucose response metric (e.g., glucose high, glucose low, maximum rate of change, etc.) and transfer that information at 1913 to meal planner software 1918. In alternative embodiments, the HCP herself or himself determines the ranking or categorization of meals by glucose response.

Meal planner software 1918 can also access the information in user meal choice software 1914 via information path 1908. Meal planner software 1918 can determine and generate a daily, weekly, or monthly menu for the user by selecting meals that produce the relative “best” or optimal glucose responses. Based on the chosen meals, meal planner software 1918 can produce a grocery shopping list for the user having the necessary items to make the chosen meals. Meal planner software 1918 can also include an option in the menu to include restaurant meals when generally convenient or planned, e.g., a work day lunch Monday through Friday at a local restaurant or cafeteria, etc. To the extent meal planner software 1918 selects meals from nutrition database 1916 (via information path 1917), these meal selections can be based on nutritional analysis performed by meal planner software 1918 in conjunction with heuristics.

All of this information generated by meal planner software 1918 can be output and transferred to mobile software 1900, for example meal monitor application 1902, via information path 1909. Meal monitor application 1902 can include a feature or option, displayable to the user, that includes the generated daily, weekly, or monthly menu along with the associated grocery list for use by the user. In this manner, a comprehensive meal or menu planning software can be implemented that relies heavily on the data and information collected by the user through the meal monitor application 1902.

The embodiments described herein are reiterated and/or supplemented in the following paragraphs which do so without explicit reference to the figures. In many example embodiments, a method of determining an insulin dose for administration with the consumption of a meal is provided, where the method includes: compiling a database of meal records in a non-transitory memory, where each meal record in the database corresponds to a meal previously consumed by a user and includes an insulin dose administered with that previously consumed meal, and where each meal record in the database is classified as one of a plurality of menu items; inputting, by the user into an electronic device, a selection of a menu item from one of the plurality of menu items; retrieving, by the electronic device, information from each of the meal records classified as the selected menu item; and recommending, by the electronic device, an insulin dose based on the retrieved information.

In some embodiments, each meal record in the database does not include a carbohydrate content of the meal and recommending, by the electronic device, the insulin dose based on the retrieved information is performed without reference to a carbohydrate content of the selected menu item.

In some embodiments, the retrieved information includes the most recently administered insulin dose for the selected menu item, and the method including recommending, by the electronic device, the insulin dose based only on the most recently administered insulin dose and one or more correction parameters.

In some embodiments, recommending, by the electronic device, the insulin dose based on the retrieved information includes: determining, by the electronic device, the recommended insulin dose; and displaying the recommended insulin dose to the user on a display of the electronic device along with the user's glucose data. The user's glucose data can be displayed on a graph with additional glucose data corresponding to one or more previous instances of consumption of the meal of the selected menu item. Each of the meal records classified as the selected menu item can include glucose data from a time period of the corresponding meal.

In some embodiments, the electronic device is a reader device, and the method further including reading in vivo glucose data of the user, by the reader device, from a sensor control device on the body of the user.

In some embodiments, the recommended insulin dose includes at least two dose components, and the recommended insulin dose is displayed to the user with the at least two dose components. The method can also include receiving a modification, by the electronic device, to at least one of the at least two dose components and updating the recommended insulin dose based on the received modification to the at least one of the at least two dose components.

In some embodiments, one or more of the meal records further include: a description of the meal; a time of consumption of the meal; and glucose data of the user from a time period occurring before and after the time of consumption of the meal. The one or more of the meal records can further include an image of the meal or the menu item.

In some embodiments, the selection of the menu item is input to a meal selection screen displayed on the electronic device, and the meal selection screen includes the plurality of menu items and an indication of the frequency that each of the plurality of menu items has previously been consumed by the user. Also, in many embodiments the method includes administering the recommended insulin dose.

In many embodiments an electronic system configured to determine an insulin dose for administration with the consumption of a meal is provided, the system including: processing circuitry; and a non-transitory memory including a plurality of instructions that, when executed, cause the processing circuitry to: read a selection of a menu item from one of a plurality of menu items; retrieve information from a database of meal records, where each meal record in the database corresponds to a meal previously consumed by a user and includes an insulin dose administered with that previously consumed meal, and where each meal record in the database is classified as one of the plurality of menu items, the retrieved information being from each of the meal records classified as the selected menu item; and determine a recommendation of an insulin dose based on the retrieved information.

In some embodiments, each meal record in the database does not include a carbohydrate content of the meal and the plurality of instructions, when executed, cause the processing circuitry to determine the recommendation of the insulin dose based on the retrieved information and without reference to a carbohydrate content of the selected menu item.

In some embodiments, the retrieved information includes the most recently administered insulin dose for the selected menu item, and the plurality of instructions, when executed, cause the processing circuitry to determine the recommendation of the insulin dose based only on the most recently administered insulin dose and one or more correction parameters. The plurality of instructions, when executed, can also cause the processing circuitry to display the recommended insulin dose to the user on an electronic display of the system along with the user's glucose data, and the user's glucose data can be displayed in a graph with additional glucose data corresponding to one or more previous instances of consumption of the meal of the selected menu item. In some embodiments, each of the meal records classified as the selected menu item includes glucose data from a time period of the corresponding meal.

In some embodiments, the system includes a reader device and a sensor control device including an in vivo sensor, where the reader device is configured to read glucose data from the sensor control device.

In some embodiments, the recommended insulin dose includes at least two dose components, and the plurality of instructions, when executed, cause the processing circuitry to display the recommended insulin dose to the user with the at least two dose components. The plurality of instructions, when executed, can also cause the processing circuitry to read a modification, entered by the user, to at least one of the at least two dose components and update the recommended insulin dose based on the modification to the at least one of the at least two dose components.

In some embodiments, one or more of the meal records further include: a description of the meal; a time of consumption of the meal; and glucose data of the user from a time period occurring before and after the time of consumption of the meal. The one or more of the meal records can further include an image of the meal or the menu item.

In some embodiments, the plurality of instructions, when executed, cause the processing circuitry to display a meal selection screen on a touch screen display of the system, the meal selection screen including the plurality of menu items and an indication of the frequency that each of the plurality of menu items has previously been consumed by the user.

In some embodiments, the processing circuitry and non-transitory memory are components of a smartphone of the system and the database is stored on the non-transitory memory of the smartphone. The system can further include an insulin delivery device to administer to insulin dose.

In many embodiments, a method of determining an insulin dose for administration with the performance of an activity is provided, the method including: compiling a database of activity records in a non-transitory memory, where each activity record in the database corresponds to an activity previously performed by a user and includes an insulin dose administered with that previously performed activity, and where each activity record in the database is classified as one of a plurality of activity types; inputting, by the user into an electronic device, a selection of an activity type from one of the plurality of activity types; retrieving, by the electronic device, information from each of the activity records classified as the selected activity type; and recommending, by the electronic device, an insulin dose based on the retrieved information.

In many embodiments, an electronic system configured to determine an insulin dose for administration with the performance of an activity is provided, the system including: processing circuitry; and a non-transitory memory including a plurality of instructions that, when executed, cause the processing circuitry to: read a selection of an activity type from one of a plurality of activity types; retrieve information from a database of activity records, where each activity record in the database corresponds to an activity previously performed by a user and includes an insulin dose administered with that previously performed activity, and where each activity record in the database is classified as one of the plurality of activity types, the retrieved information being from each of the activity records classified as the selected activity type; and determine a recommendation of an insulin dose based on the retrieved information.

In many embodiments, a system for menu planning is provided, including: a mobile device including first processing circuitry and a first non-transitory memory including a first plurality of instructions that, when executed, cause the first processing circuitry to: collect analyte data from a user; and associate the analyte data with meal information entered by the user; and a cloud-based system including second processing circuitry and a second non-transitory memory including a second plurality of instructions that, when executed, cause the second processing circuitry to: access the collected analyte data and associated meal information of the user for multiple meals consumed by the user; identify a plurality of meals that are associated with relatively optimal analyte responses by the user; generate a menu for the user including the identified plurality of meals; and output the generated menu to the mobile device.

In some embodiments, the second plurality of instructions, when executed, cause the second processing circuitry to: generate a grocery list for the user including ingredients to make the identified plurality of meals; and output the generated grocery list to the mobile device.

In some embodiments, the second plurality of instructions, when executed, cause the second processing circuitry to: access a nutritional database including nutritional information about a plurality of restaurant meals identified by the user; and generate the menu for the user including the identified plurality of meals at least one restaurant meal.

In some embodiments, the second plurality of instructions, when executed, cause the second processing circuitry to identify the plurality of meals that are associated with relatively optimal analyte responses by the user based on a categorization of meal responses received from a health care professional.

In some embodiments, the first plurality of instructions, when executed, cause the first processing circuitry to upload the collected analyte data and associated meal information to the cloud-based system. In some embodiments, the mobile device is configured to collect analyte data by reading in vivo analyte data from a sensor control device on the body of the user.

In many embodiments, a method of menu planning is provided, where the method includes: collecting, by a mobile device, analyte data from a user; associating, by the mobile device, the analyte data with meal information entered by the user; outputting the collected analyte data and associated meal information of the user to a cloud-based system; identifying, by the cloud-based system, a plurality of meals that are associated with relatively optimal analyte responses by the user; generating, by the cloud-based system, a menu for the user including the identified plurality of meals; and outputting the generated menu to the mobile device.

In some embodiments, the method includes generating, by the cloud-based system, a grocery list for the user including ingredients to make the identified plurality of meals and outputting the generated grocery list to the mobile device.

In some embodiments, the method includes accessing a nutritional database including nutritional information about a plurality of restaurant meals identified by the user; and generating, by the cloud-based system, the menu for the user including the identified plurality of meals and at least one restaurant meal.

In some embodiments, the plurality of meals that are associated with relatively optimal analyte responses by the user are based on a categorization of meal responses received from a health care professional. In some embodiments, collecting, by the mobile device, analyte data from a user includes reading, by the mobile device, in vivo glucose data from a sensor control device on the body of the user.

To the extent information is input by a user, device, or other software, then that information can be received, read, and if containing an instruction, implemented by the processing circuitry (which can be a single processor or distributed across multiple processors or devices having processing capability). Those instructions can be stored in a memory of, and executed by processing circuitry of, any and all embodiments of a reader device, drug delivery device, meter, computer system, or other computing device described herein.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

In many instances entities are described herein as being coupled to other entities. It should be understood that the terms “coupled” and “connected” (or any of their forms) are used interchangeably herein and, in both cases, are generic to the direct coupling of two entities (without any non-negligible intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

The subject matter described herein and in the accompanying figures is done so with sufficient detail and clarity to permit the inclusion of claims, at any time, in means-plus-function format pursuant to 35 U.S.C. section 112, part (f). However, a claim is to be interpreted as invoking this means-plus-function format only if the phrase “means for” is explicitly recited in that claim.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

While the embodiments are susceptible to various modifications and alternative forms, specific examples thereof have been shown in the drawings and are herein described in detail. These embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the scope of the claims by features, functions, steps, or elements that are not within that scope.

1. A method of determining an insulin dose for administration with the consumption of a meal, the method comprising:
compiling a database of meal records in a non-transitory memory, wherein each meal record in the database corresponds to a meal previously consumed by a user and comprises an insulin dose administered with that previously consumed meal, and wherein each meal record in the database is classified as one of a plurality of menu items;
inputting, by the user into an electronic device, a selection of a menu item from one of the plurality of menu items;
retrieving, by the electronic device, information from each of the meal records classified as the selected menu item; and
recommending, by the electronic device, an insulin dose based on the retrieved information.

2. The method of claim 1, wherein each meal record in the database does not comprise a carbohydrate content of the meal.

3. The method of claim 2, comprising recommending, by the electronic device, the insulin dose based on the retrieved information and without reference to a carbohydrate content of the selected menu item.

4. The method of claim 2, wherein the retrieved information comprises the most recently administered insulin dose for the selected menu item, the method comprising recommending, by the electronic device, the insulin dose based only on the most recently administered insulin dose and one or more correction parameters.

5. The method of claim 1, wherein recommending, by the electronic device, the insulin dose based on the retrieved information comprises:
determining, by the electronic device, the recommended insulin dose; and
displaying the recommended insulin dose to the user on a display of the electronic device along with the user's glucose data.

6. The method of claim 5, wherein the user's glucose data is displayed on a graph with additional glucose data corresponding to one or more previous instances of consumption of the meal of the selected menu item.

7. The method of claim 6, wherein each of the meal records classified as the selected menu item comprises glucose data from a time period of the corresponding meal.

8. The method of claim 5, wherein the electronic device is a reader device, the method further comprising reading in vivo glucose data of the user, by the reader device, from a sensor control device on the body of the user.

9. The method of claim 5, wherein the recommended insulin dose comprises at least two dose components, and wherein the recommended insulin dose is displayed to the user with the at least two dose components.

10. The method of claim 9, further comprising receiving a modification, by the electronic device, to at least one of the at least two dose components.

11. The method of claim 10, further comprising updating the recommended insulin dose based on the received modification to the at least one of the at least two dose components.

12. The method of claim 1, wherein one or more of the meal records further comprise:
a description of the meal;
a time of consumption of the meal; and
glucose data of the user from a time period occurring before and after the time of consumption of the meal.

13. The method of claim 12, wherein the one or more of the meal records further comprise an image of the meal or the menu item.

14. The method of claim 1, wherein the selection of the menu item is input to a meal selection screen displayed on the electronic device, and wherein the meal selection screen comprises the plurality of menu items and an indication of the frequency that each of the plurality of menu items has previously been consumed by the user.

15. The method of claim 1, further comprising administering the recommended insulin dose.

16-45. (canceled)
